(19) United States
(12) Patent Application Publication
(10) Pub. No.: US 2010/0120104 A1
(43) Pub. Date: May 13, 2010

(54) BIOLOGICAL AND CHEMICAL PROCESS UTILIZING CHEMOAUTOTROPHIC MICROORGANISMS FOR THE CHEMOSYTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS

(76) Inventor: John Stuart Reed, Sacramento, CA (US)

Correspondence Address: john reed, 614 N street, Sacramento, CA, 95814, US

(21) Appl. No.: 12/613,550

(22) Filed: Nov. 6, 2009

Related U.S. Application Data
(60) Provisional application No. 61/111,794, filed on Nov. 6, 2008.

(51) Int. Cl. C12P 7/54; C12P 1/00; C12P 1/04; C12P 7/40; C12P 7/06; C12P 7/16; C12P 5/02; C12P 5/00; C12P 3/00

(52) U.S. Cl. 435/140; 435/41; 435/170; 435/136; 435/161; 435/160; 435/167; 435/166; 435/168

(57) ABSTRACT

The invention described herein presents compositions and methods for a multistep biological and chemical process for the capture and conversion of carbon dioxide and/or other forms of inorganic carbon into organic chemicals including biofuels or other useful industrial, chemical, pharmaceutical, or biomass products. One or more process steps in the present invention utilizes chemoautotrophic microorganisms to fix inorganic carbon into organic compounds through chemosynthesis. An additional feature of the present invention describes process steps whereby electron donors used for the chemosynthetic fixation of carbon are generated by chemical or electrochemical means, or are produced from inorganic or waste sources. An additional feature of the present invention describes process steps for the recovery of useful chemicals produced by the carbon dioxide capture and conversion process, both from chemosynthetic reaction steps, as well as from non-biological reaction steps.

US Non-Provisional application for Utility patent, Submitted on Nov. 4, 2009 Claims priority of provisional application 61111794, filed on Nov. 6, 2008

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### FIELD OF THE INVENTION

The present invention falls within the technical areas of biofuels, bioremediation, carbon capture, carbon dioxide-to-fuels, carbon recycling, carbon sequestration, energy storage, and renewable/alternative and/or low carbon dioxide emission sources of energy. Specifically the present invention is a unique example of the use of biocatalysts within a biological and chemical process to fix carbon dioxide and/or other forms of inorganic carbon into organic chemical products through chemosynthesis. In addition the present invention involves the production of chemical co-products that are co-generated through chemosynthetic reaction steps and/or non-biological reaction steps as part of an overall carbon capture and conversion process. The present invention enables the economic capture of carbon dioxide from the atmosphere or from a point source of carbon dioxide emissions for the production of liquid transportation fuel and/or other organic chemical products, which will help address greenhouse gas induced climate change and contribute to the domestic production of renewable liquid transportation fuels without any dependence upon agriculture.

### BACKGROUND OF THE INVENTION

The amazing technological and economic progress achieved in the past 100 years has largely been powered by fossil fuels. However the sustainability of this progress is now coming into question, both due to the rise in greenhouses gases caused by fossil fuel combustion, and the increasing scarcity of fossil fuel resources.

The increasing carbon dioxide (CO₂) concentrations in the atmosphere due to human activity are broadly acknowledged to be one of the major causes of climate change. Changes in climate being observed, which are projected to increase in severity over time, include global-warming, carbon cycle disturbances, and the melting of Antarctic and Arctic polar ice caps. [Vital Climate Change Graphics, United Nations Environmental Programme, February 2005]. The use of fossil fuels is a major factor in anthropogenic climate change since fossil fuel combustion adds carbon dioxide, and greenhouse gases such as nitric oxide into the atmosphere. Over 30 billion metric tons of carbon dioxide are emitted world-wide every year by human activities and the emission trend is on the rise. [Energy Information Administration, 2008]. This makes climate change one of the most serious environmental issues with potentially disruptive social and economic consequences [IPCC, 2007]

Governments have begun to act to mitigate greenhouse gases and to reduce their potential impacts. The Kyoto Protocol, the Boxer-Lieberman-Warner bill, the Western Climate Initiative (WCI) and the California Assembly Bill 32 (AB32) all show that there is a commitment to reducing greenhouse gas levels. The global market for technology solutions to reduce CO₂ emissions is predicted to grow to $236 B by 2012 (ClimatBiz, 2008) reaching $400 B by 2030 (BER, 2008).

The use of fossil petroleum humanity's chief source of liquid transportation fuel brings a host of additional problems beyond the contribution to climate change. These problems are connected to the increasing scarcity of petroleum resources and the political instabilities widely associated with oil producing nations. Transitioning away from petroleum, and fossil fuels in general, is a major challenge due to the essential role fossil fuels play in powering the world economy, and the large infrastructure that has been put in place for their use.

Efforts to technologically address the problem of carbon dioxide emissions posed by the use of fossil fuels have developed along three main lines: improving energy efficiency; carbon capture and sequestration/recycling; developing alternative energy systems that are renewable and/or have low- or no-CO₂ emissions.

Renewable and/or carbon emission-free alternative energy technologies subject to ongoing research and development can generally be categorized as either based on inorganic processes or on biological processes. Those based on inorganic processes include photovoltaics, solar thermal, wind power, hydroelectric, geothermal, fuel cells, and batteries [Global Trends in Sustainable Energy Investment 2007, United Nations Environmental Programme].

Hydrogen which can be generated through a number of different inorganic renewable energy technologies including solar, wind, and geothermal has been proposed as a replacement for hydrocarbon fuels. But hydrogen has its own set of problems including most notably problems with storage. Ironically the best chemical storage medium for hydrogen both in terms of volumetric and gravimetric energy densities is quite possibly hydrocarbons such as gasoline, suggesting that the quest for hydrogen fuel may simply lead full circle back to hydrocarbons.

Most biologically based alternative energy technologies focus on the creation of biofuels. Biofuels are generally made through the capture and conversion of CO₂ via photosynthesis into organic matter. This organic product of photosynthesis generally needs to be further processed biologically or chemically to become a biofuel such as biodiesel, ethanol, renewable diesel or gasoline. Since the current transportation fleet and infrastructure is designed for fossil fuels with similar properties to biofuels, it can be more readily be adapted to biofuels, than to inorganic energy storage products such as hydrogen or batteries. A further advantage of biofuels, and hydrocarbons in general, is that they have some of the highest volumetric and gravimetric energy densities found for any form of chemical energy storage—substantially higher than that achieved with current lithium battery and hydrogen storage technologies. However, biofuels produced through photosynthesis have its own set of problems.

Most biofuel currently produced relies on agriculture. The heavy requirements of large scale agricultural biofuel projects for arable land, fresh water, and other resources required for plant growth have been blamed for rapidly increasing food prices and loss of natural habitat [The Price of Biofuels: The Economics Behind Alternative Fuels, Technology Review, January/February 2008].

The drawbacks to the agricultural production of biofuels, and non-food products generally, from CO₂ through photosynthesis, can be summarized as follows:
1. Food versus fuel competition
2. Heavy water use
3. Fertilizer, herbicide, and/or pesticide run-off
4. Deforestation
5. Loss of natural habitat

As an alternative to higher order plants, photosynthetic microorganisms such as algae and cyanobacteria are being looked at for applications converting CO₂ into biofuels or other organic chemicals [Sheehan et al, 1998, “A Look Back at the U.S. Department of Energy's Aquatic Species Program—Biodiesel from Algae”]. As with higher order plants, the products of recycling CO₂ are relatively valuable (e.g. algae cake, biofuel or biofuel feedstock). Algal and cyanobacterial technologies also benefit from the relatively high growth rates of photosynthetic microbes which can far surpass higher order plants in their rate of carbon fixation per unit standing biomass. In one promising application of algal technology a high rate of carbon fixation and biomass production is achieved by directing a concentrated stream of CO₂, such as is emitted from industrial point sources, through algae containing bioreactors [Bayless et al. U.S. Pat. No. 6,667,171].

Technologies based on photosynthetic microbes share the drawback common to all photosynthetic systems in that carbon fixation only happens with light exposure. Therefore these technologies can only capture carbon during daylight hours having sufficient sunlight, unless artificial lighting is made available during nighttime or cloudy weather. The use of artificial lighting has the downside of being an additional energy drain and a source of additional CO₂ emissions (unless an emission-free source of electricity is available). If the light level is deficient, an algal system can actually become a net producer of CO₂ emissions. It is often optimal to run many CO₂ emitting industrial operations continually—day and night, in all weather and seasons. For these types of operations an algae technology that captures CO₂ only when sufficient sunlight is present will not be able to capture the majority of CO₂ emissions. Similarly light requirements can limit the geographical range for the practical application of algal technologies to areas having enough sunlight.

A bioreactor or pond used to grow photosynthetic microbes such as algae must have a high surface area to volume ratio in order to allow each cell to receive enough light for carbon fixation and cell growth. Otherwise light blockage by cells on the surface will leave cells located towards the center of the volume in darkness—turning them into net CO₂ emitters. This high surface area to volume ratio needed for efficient implementation of the algal and cyanobacterial technologies generally results in either a large land footprint (ponds) or high material costs (bioreactors). The types of materials that can be used in algal bioreactor construction is limited by the requirement that walls lying between the light source and the algal growth environment need to be transparent. This requirement restricts the use of construction materials that would normally be preferred for use in large scale projects such as concrete, steel and earthworks.

The downside of technologies for the capture and recycling of carbon dioxide that rely on photosynthetic microbes can be summarized as follows [Sheehan et al, 1998, “A Look Back at the U.S. Department of Energy's Aquatic Species Program—Biodiesel from Algae”]:
1. Limited to geographies with sufficient year-round sunlight
2. Carbon capture does not run continuously; microbes emit CO₂ when light is not present
3. Ponds have the most favorable economics, but only approximately 6 places on the planet provide the optimal conditions for pond growth of photosynthetic microbes
4. Ponds most suitable for algal growth are wide and shallow (˜10 cm deep) in order to maximize light exposure leading to a large land area footprint
5. Growth in bioreactors designed to reduce the land footprint has proven difficult to scale since it requires novel, high surface area reactor architectures (e.g. thin, flat sheet or narrow tubular structures) and construction out of transparent materials [Bayless et al. U.S. Pat. No. 6,667,171]. Schemes involving solar collectors or light guiding pipes are also being attempted but have yet to prove practical.
6. Conventional bioreactors used in large scale microbial processes such as enzyme production and wastewater or sewage treatment are not appropriate for algal growth due to their relatively deep tanks (5-10 m) and construction from opaque materials such as concrete and steel.
7. Many of the constituents of industrial flue gas are poisonous to algae, limiting applicability and requiring cleaning of flue stream

As has been discussed, most of the current CO₂ abatement technologies show several limitations. However the EPA in the report “Climate Change Scoping Plan” predicts that carbon capture technologies will have a very important role to play in the future “The Economic and Technology Advancement Advisory Committee recognized the importance of pursuing technologies that are transformative in nature. Two of the technologies that they highlighted are “smart grids” and carbon capture and sequestration” [C-EPA, 2008].

In addition to the biological CO2 fixation processes that have been discussed, there are also fully chemical processes for fixing CO2 to organic compounds (LBNL Helios; LANL Green Freedom; Sandia Sunshine to Petrol; PARC). The fully chemical technologies are currently hindered by the catalysts that are needed for the relatively complicated reaction of CO2 to fixed carbon, especially C2 and longer hydrocarbons. Due to the lack of adequate catalysts the fully chemical CO2-to-fuel technologies are generally at an early stage of development. For example Sandia's Sunshine to Petrol program is reported to be about 15 to 20 years away from market.

Chemoautotrophic microorganisms represent a possible alternative to photosynthetic organisms for use in carbon fixation processes that can avoid the shortcomings of photosynthesis discussed above, while still leveraging billions of years of enzymatic evolution for catalyzing the carbon fixation reaction. The chemosynthetic reactions performed by chemoautotrophs for the fixation of CO2, and other forms of inorganic carbon, to organic compounds, is powered by potential energy stored in inorganic chemicals, rather than by the radiant energy of light [Shively et al, 1998; Smith et al, 1967; Hugler et al, 2005; Hugker et al., 2005; Scott and Cavanaugh, 2007]. Carbon fixing biochemical pathways that occur in chemoautotrophs include the reductive tricarboxylic acid cycle, the Calvin-Benson-Bassham cycle [Jessup Shively, Geertje van Kaulen, Wim Meijer, Annu Rev. Microbiol., 1998, 191-230], and the Wood-Ljungdahl pathway [Ljungdahl, 1986; Gottschalk, 1989; Lee, 2008; Fischer, 2008].

An extensive search of the prior art reveals that there are prior inventions that have claimed applications of chemoautotrophic microorganisms in the capture and conversion of CO2 gas to fixed carbon. Some particularly relevant inventions are: [U.S. Pat. No. 4,596,778 “Single cell protein from sulfur energy sources” Hitzman, Jun. 24, 1986], [U.S. Pat. No. 4,859,588 “Production of a single cell protein”, Sublette Aug. 22, 1989], [U.S. Pat. No. 5,593,886 “Clostridium strain which produces acetic acid from waste gases Gaddy”, Jan. 14, 1997], [U.S. Pat. No. 5,989,513 “Biologically assisted process for treating sour gas at high pH”, Rai Nov. 23, 1999]. The present invention described herein has novel aspects, and important distinctions and differences with the past inventions using chemoautotrophs for CO2 capture, which it is believed will lead to wide spread use of the present invention for CO2 capture for biofuel and/or organic chemical production, whereas these past inventions have had limited practical application.

Chemoautotrophic microorganisms have also been used to biologically convert syngas into C2 and longer organic compounds including acetic acid and acetate, and biofuels such as ethanol and butanol [Gaddy, 2007; Lewis, 2007; Heiskanen, 2007; Worden, 1991; Klasson, 1992; Ahmed, 2006; Cotter, 2008; Piccolo, 2008, Wei, 2008]. While biological syngas-to-biofuel conversions have some similarities with the present invention, the applications and overall process are fundamentally different. In syngas conversions to biofuel, the feedstock is fixed carbon (either biomass or fossil fuel), which is gasified and then biologically converted to another form of fixed carbon—biofuel. The present invention described herein by contrast does not require any fixed carbon feedstock, only CO₂ and/or other forms of inorganic carbon. The carbon fixation of inorganic carbon occurs within the present invention, not prior to the process as with syngas to biofuel conversions. In syngas to biofuel conversions the carbon source and energy source come from the same process input, either biomass or fossil fuel, and are completely intermixed within the syngas in the form of H₂, CO, and CO₂. In contrast, for the present invention, the carbon source and the energy source are separate process inputs.

This separation of carbon source from energy source enables the present invention to function as a far more general energy storage technology than syngas to liquid fuel conversions. This is because the electron donors used in the present invention can be generated from a wide array of different CO₂-free energy sources, both conventional and alternative, while for syngas conversions to biofuel, all the energy stored in the biofuel is ultimately derived from photosynthesis (with additional geochemical energy in the case of fossil fuel feedstock).

It is worth noting that various types of chemoautotrophs have found practical application in the field of bioremediation for the uptake and conversion of environmental contaminants and pollutants other than carbon dioxide, such as heavy metals (Cr, Mn), hydrocarbons, halogenated hydrocarbons, nitrates, nitrous oxide, and radioactive materials. Patented inventions that use chemoautotrophs for the absorption of nitrous oxide from flue gases [U.S. Pat. No. 5,077,208] are also relevant to the present invention since the present invention applies chemoautotrophs to the remediation of flue gas emissions, albeit to carbon dioxide rather than nitrous oxide.

### SUMMARY OF THE INVENTION

In response to a need in the art the present invention provides a novel combined biological and chemical process for the capture and conversion of inorganic carbon to organic compounds that uses chemosynthetic microorganisms for carbon fixation and that is designed to couple the efficient production of high value organic compounds such as liquid hydrocarbon fuel with the capture of CO₂ emissions, making carbon capture a revenue generating process.

The present invention gives compositions and methods for the capture of carbon dioxide from carbon dioxide-containing gas streams and/or atmospheric carbon dioxide or carbon dioxide in dissolved, liquefied or chemically-bound form through a chemical and biological process that utilizes obligate or facultative chemoautotrophic microorganisms and particularly chemolithoautotrophic organisms, and/or cell extracts containing enzymes from chemoautotrophic microorganisms in one or more carbon fixing process steps. The present invention also gives compositions and methods for the recovery, processing, and use of the chemical products of chemosynthetic reactions performed by chemoautotrophs to fix inorganic carbon into organic compounds. The present invention also gives compositions and methods for the generation, processing and delivery of chemical nutrients needed for chemosynthesis and maintenance of chemoautotrophic cultures, including but not limited to the provision of electron donors and electron acceptors needed for chemosynthesis. The present invention also gives compositions and methods for the maintenance of an environment conducive for chemosynthesis and chemoautotrophic growth, and the recovery and recycling of unused chemical nutrients and process water.

The present invention also gives compositions and methods for chemical process steps that occur in series and/or in parallel with the chemosynthetic reaction steps that: convert unrefined raw input chemicals to more refined chemicals that are suited for supporting the chemosynthetic carbon fixing step; that convert energy inputs into a chemical form that can be used to drive chemosynthesis, and specifically into chemical energy in the form of electron donors and electron acceptors; that direct inorganic carbon captured from industrial or atmospheric or aquatic sources to the carbon fixation steps of the process under conditions that are suitable to support chemosynthetic carbon fixation; that further process the output products of the chemosynthetic carbon fixation steps into a form suitable for storage, shipping, and sale, and/or safe disposal in a manner that results in a net reduction of gaseous CO2 released into the atmosphere. The fully chemical process steps combined with the chemosynthetic carbon fixation steps constitute the overall carbon capture and conversion process of the present invention. The present invention utilizes the unique ease of integrating chemoautotrophic microorganisms into a chemical process stream as a biocatalyst, as compared to other lifeforms. This unique capability arises from the fact that chemoautotrophs naturally act at the interface of biology and chemistry through their chemosynthetic lifestyle.

One feature of the present invention is the inclusion of one or more process steps within a chemical process for the capture of inorganic carbon and conversion to fixed carbon products, that utilize chemoautotrophic microorganisms and/or enzymes from chemoautotrophic microorganisms as a biocatalyst for the fixation of carbon dioxide in carbon dioxide-containing gas streams or the atmosphere or water and/or dissolved or solid forms of inorganic carbon, into organic compounds. In these process steps carbon dioxide containing flue gas, or process gas, or air, or inorganic carbon in solution as dissolved carbon dioxide, carbonate ion, or bicarbonate ion including aqueous solutions such as sea water, or inorganic carbon in solid phases such as but not limited to carbonates and bicarbonates, is pumped or otherwise added to a vessel or enclosure containing nutrient media and chemoautotrophic microorganisms. In these process steps chemoautotrophic microorganisms perform chemosynthesis to fix inorganic carbon into organic compounds using the chemical energy stored in one or more types of electron donor pumped or otherwise provided to the nutrient media including but not limited to one of more of the following: ammonia; ammonium; carbon monoxide; dithionite; elemental sulfur; hydrocarbons; hydrogen; metabisulfites; nitric oxide; nitrites; sulfates such as thiosulfates including but not limited to sodium thiosulfate (Na.sub.2S.sub.2O.sub.3) or calcium thiosulfate (CaS.sub.2O.sub.3); sulfides such as hydrogen sulfide; sulfites; thionate; thionite; transition metals or their sulfides, oxides, chalcogenides, halides, hydroxides, oxyhydroxides, sulfates, or carbonates, in soluble or solid phases; as well as valence or conduction electrons in solid state electrode materials. The electron donors are oxidized by electron acceptors in the chemosynthetic reaction. Electron acceptors that may be used as the chemosynthetic reaction step include but are not limited to one or more of the following: carbon dioxide, ferric iron or other transition metal ions, nitrates, nitrites, oxygen, sulfates, or holes in solid state electrode materials.

The chemosynthetic reaction step or steps of the process whereby carbon dioxide and/or inorganic carbon is fixed into organic carbon in the form of organic compounds and biomass can be performed in aerobic, microaerobic, anoxic, anaerobic, or facultative conditions. A facultative environment is considered to be one where the water column is stratified into aerobic layers and anaerobic layers. The oxygen level maintained spatially and temporally in the system will depend upon the chemoautotrophic species used, and the desired chemosynthesis reactions to be performed.

Additional carbon dioxide may be sequestered in process steps occurring in series or parallel to the chemosynthetic process steps where carbon dioxide is reacted with minerals including but not limited to oxides or hydroxides to form a carbonate or bicarbonate product. Additional carbon may also be sequestered into solid carbonates through process steps occurring in series or in parallel to the chemosynthetic process steps where chemical reactions are performed that generate or recycle electron donor chemicals used in the chemosynthetic process step/s including but not limited to oxidation of hydrocarbons or coal by sulfate minerals to form sulfide electron donors and solid carbonate products. Further carbon dioxide may be sequestered through the catalytic action of chemoautotrophic microorganisms that convert carbon dioxide into inorganic carbonates or biominerals within in the chemosynthetic process step/s.

An additional feature of the present invention regards the source, production, or recycling of the electron donors used by the chemoautotrophic microorganisms to fix carbon dioxide into organic compounds. The electron donors used for carbon dioxide capture and carbon fixation can be produced or recycled in the present invention electrochemically or thermochemically using power from a number of different renewable and/or low carbon emission energy technologies including but not limited to: photovoltaics, solar thermal, wind power, hydroelectric, nuclear, geothermal, enhanced geothermal, ocean thermal, ocean wave power, tidal power. The electron donors can also be of mineralogical origin including but not limited to reduced S and Fe containing minerals. The present invention enables the use of a largely untapped source of energy—inorganic geochemical energy. The electron donors used in the present invention can also be produced or recycled through chemical reactions with hydrocarbons that may or may not be a non-renewable fossil fuel, but where said chemical reactions produce low or zero carbon dioxide gas emissions. Such electron donor generating chemical reactions that can be used as steps in the process of the present invention include but are not limited to: the thermochemical reduction of sulfate reaction or TSR [Evaluating the Risk of Encountering Non-hydrocarbon Gas Contaminants (CO2, N2, H2S) Using Gas Geochemistry, www.gaschem.com/evalu.html] or the Muller-Kuhne reaction; the reduction of metal oxides including iron oxide, calcium oxide, and magnesium oxide. The reaction formula for TSR is CaSO.sub.4+CH.sub.4→CaCO.sub.3+H.sub.2O+H.sub.2S. In this case the electron donor product that can be used by chemoautotrophic microorganisms for CO₂ fixation is hydrogen sulfide. The solid carbonate product also formed can be easily sequestered resulting in no release of carbon dioxide into the atmosphere. There are similar reactions reducing sulfate to sulfide that involve longer chain hydrocarbons [Changtao Yue, Shuyuan Li, Kangle Ding, Ningning Zhong, Thermodynamics and kinetics of reactions between C1-C3 hydrocarbons and calcium sulfate in deep carbonate reservoirs, Geochem. Jour., 2006, 87-94]. The Muller-Kuhne reaction formula is 2C+4CaSO₄→2CaO+2CaCO₃+4SO₂. The SO₂ produced can be further reacted with S and a base including but not limited to lime, magnesium oxide, iron oxide, or some other metal oxide to produce an electron donor such as thiosulfate (S.sub.2.O.sub.3.sup.2-) usable by chemoautotrophs. It is preferred that the base used in the reaction to form (S.sub.2.O.sub.3.sup.2-) is produced from a carbon dioxide emission-free source such as natural sources of basic minerals including but not limited to calcium oxide, magnesium oxide, olivine containing a metal oxide, serpentine containing a metal oxide, ultramafic deposits containing metal oxides, and underground basic saline aquifers. Example of oxide reduction reactions that produce a carbonate and a hydrogen product that can be used as electron donor in the chemosynthetic reaction steps of the present invention include 2CH.sub.4+Fe.sub.2O.sub.3+3H.sub.2O->2FeCO.sub.3+7H.sub.2 or CH.sub.4+CaO+2H.sub.2O->CaCO.sub.3+4H.sub.2. Since the TSR reaction and the like are exothermic, it is preferred that some of the energy released by the reaction be recovered to improve the overall energy efficiency of the process. Therefore preferred embodiments of this invention which rely on exothermic reactions such as the TSR for electron donor generation utilize the heat energy and/or electrochemical energy released by the reaction to improve the overall energy efficiency of the process.

An additional feature of the present invention regards the formation and recovery of useful organic and/or inorganic chemical products from the chemosynthetic reaction step or steps including but not limited to one or more of the following: acetic acid, other organic acids and salts of organic acids, ethanol, butanol, methane, hydrogen, hydrocarbons, sulfuric acid, sulfate salts, elemental sulfur, sulfides, nitrates, ferric iron and other transition metal ions, other salts, acids or bases. These chemical products can be applied to uses including but not limited to one or more of the following: as a fuel; as a feedstock for the production of fuels; in the production of fertilizers; as a leaching agent for the chemical extraction of metals in mining or bioremediation; as chemicals reagents in industrial or mining processes.

An additional feature of the present invention regards the formation and recovery of biochemicals and/or biomass from the chemosynthetic carbon fixation step or steps. These biochemical and/or biomass products can have applications including but not limited to one or more of the following: as a biomass fuel for combustion in particular as a fuel to be co-fired with fossil fuels such as coal in pulverized coal powered generation units; as a carbon source for large scale fermentations to produce various chemicals including but not limited to commercial enzymes, antibiotics, amino acids, vitamins, bioplastics, glycerol, or 1,3-propanediol; as a nutrient source for the growth of other microbes or organisms; as feed for animals including but not limited to cattle, sheep, chickens, pigs, or fish; as feed stock for alcohol or other biofuel fermentation and/or gasification and liquefaction processes including but not limited to direct liquefaction, Fisher Tropsch processes, methanol synthesis, pyrolysis, or microbial syngas conversions, for the production of liquid fuel; as feed stock for methane or biogas production; as fertilizer; as raw material for manufacturing or chemical processes such as but not limited to the production of biodegradable/biocompatible plastics; as sources of pharmaceutical, medicinal or nutritional substances; soil additives and soil stabilizers.

An additional feature of the present invention regards using modified chemoautotrophic microorganisms in the chemosynthesis process step/steps such that a superior quantity and/or quality of organic compounds, biochemicals, or biomass is generated through chemosynthesis. The chemoautotrophic microbes used in these steps may be modified through artificial means including but not limited to accelerated mutagenesis (e.g. using ultraviolet light or chemical treatments), genetic engineering or modification, hybridization, synthetic biology or traditional selective breeding. Possible modifications of the chemoautotrophic microorganisms include but are not limited to those directed at producing increased quantity and/or quality of organic compounds and/or biomass to be used as a biofuels, or as feedstock for the production of biofuels including, but not limited to biodiesel, butanol, ethanol, gasoline, hydrocarbons, methane, renewable diesel, and pseudovegetable oil or another other hydrocarbon suitable for use as a renewable/alternate fuel leading to lowered greenhouse gas emissions.

### DESCRIPTION OF THE FIGURES

FIG. 1 is a general process flow diagram for one embodiment of this invention for a carbon capture and fixation process. The CO.sub.2 containing flue gas is captured from a point source or emitter. Electron donors needed for chemosynthesis are generated from input inorganic chemicals and energy. The flue gas is pumped through bioreactors containing chemoautotrophs along with electron donors and acceptors needed to drive chemosynthesis and a medium suitable to support a chemoautotrophic culture and carbon fixation through chemosynthesis. The cell culture is continuously flowed into and out of the bioreactors. After the cell culture leaves the bioreactors the cell mass is separated from the liquid medium. Cell mass needed to replenish the cell culture population at an optimal level is recycled back into the bioreactor. Surplus cell mass is dried to form a dry biomass product. Following the cell separation step chemical products of the chemosynthetic reaction are removed from the process flow and recovered. Then any undesirable waste products that might be present are removed. Following this the liquid medium and any unused nutrients are recycled back into the bioreactors.

FIG. 2 is process flow diagram for the preferred embodiment of the present invention with capture of CO.sub.2 performed by hydrogen oxidizing chemoautotrophs resulting in the production of ethanol.

FIG. 3 shows the mass balance calculated for the preferred embodiment of the present invention reacting CO.sub.2 with H.sub.2 to produce ethanol.

FIG. 4 shows the enthalpy flow calculated for the preferred embodiment of the present invention reacting CO.sub.2 with H.sub.2 to produce ethanol.

FIG. 5 shows the energy balance calculated for the preferred embodiment of the present invention reacting CO.sub.2 with H.sub.2 to produce ethanol.

FIG. 6. is the process flow diagram for the capture of CO.sub.2 by sulfur oxidizing chemoautotrophs and production of biomass and sulfuric acid.

FIG. 7. is process flow diagram for the capture of CO.sub.2 by sulfur oxidizing chemoautotrophs and production of biomass and sulfuric acid through the chemosynthetic reaction and calcium carbonate via the Muller-Kuhne reaction.

FIG. 8 is process flow diagram for the capture of CO.sub.2 by sulfur oxidizing chemoautotrophs and production of biomass and calcium carbonate and recycling of thiosulfate electron donor via the Muller-Kuhne reaction.

FIG. 9 is process flow diagram for the capture of CO.sub.2 by sulfur and iron oxidizing chemoautotrophs and production of biomass and sulfuric acid using an insoluble source of electron donors.

FIG. 10 is process flow diagram for the capture of CO.sub.2 by sulfur and hydrogen oxidizing chemoautotrophs and production of biomass, sulfuric acid, and ethanol using an insoluble source of electron donors.

FIG. 11 is process flow diagram for the capture of CO.sub.2 by iron and hydrogen oxidizing chemoautotrophs and production of biomass, ferric sulfate, carbonate and ethanol using coal or another hydrocarbon to generate electron donors in a process that does not emit gaseous CO.sub.2 emissions.

### DETAILED DESCRIPTION

The present invention provides compositions and methods for the capture and fixation of carbon dioxide from carbon dioxide-containing gas streams and/or atmospheric carbon dioxide or carbon dioxide in liquefied or chemically-bound form through a chemical and biological process that utilizes obligate or facultative chemoautotrophic microorganisms and particularly chemolithoautotrophic organisms, and/or cell extracts containing enzymes from chemoautotrophic microorganisms in one or more process steps. Cell extracts include but are not limited to: a lysate, extract, fraction or purified product exhibiting chemosynthetic enzyme activity that can be created by standard methods from chemoautotrophic microorganisms. In addition the present invention provides compositions and methods for the recovery, processing, and use of the chemical products of chemosynthetic reaction step or steps performed by chemoautotrophs to fix inorganic carbon into organic compounds. Finally the present invention provides compositions and methods for the production and processing and delivery of chemical nutrients needed for chemosynthesis and chemoautotrophic growth, and particularly electron donors and acceptors to drive the chemosynthetic reaction; compositions and methods for the maintenance of a environment conducive for chemosynthesis and chemoautotrophic growth; and compositions and methods for the removal of the chemical products of chemosynthesis from the chemoautotrophic growth environment and the recovery and recycling of unused of chemical nutrients.

The genus of chemoautotrophic microorganisms that can be used in one or more process steps of the present invention include but are not limited to one or more of the following: Acetoanaerobium sp., Acetobacterium sp., Acetogenium sp., Achromobacter sp., Acidianus sp., Acinetobacter sp., Actinomadura sp., Aeromonas sp., Alcaligenes sp., Alcaliqenes sp., Arcobacter sp., Aureobacterium sp., Bacillus sp., Beggiatoa sp., Butyribacterium sp., Carboxydothermus sp., Clostridium sp., Comamonas sp., Dehalobacter sp., Dehalococcoide sp., Dehalospirillum sp., Desulfobacterium sp., Desulfomonile sp., Desulfotomaculum sp., Desulfovibrio sp., Desulfurosarcina sp., Ectothiorhodospira sp., Enterobacter sp., Eubacterium sp., Ferroplasma sp., Halothibacillus sp., Hydrogenobacter sp., Hydrogenomonas sp., Leptospirillum sp., Metallosphaera sp., Methanobacterium sp., Methanobrevibacter sp., Methanococcus sp., Methanosarcina sp., Micrococcus sp., Nitrobacter sp., Nitrosococcus sp., Nitrosolobus sp., Nitrosomonas sp., Nitrosospira sp., Nitrosovibrio sp., Nitrospina sp., Oleomonas sp., Paracoccus sp., Peptostreptococcus sp., Planctomycetes sp., Pseudomonas sp., Ralstonia sp., Rhodobacter sp., Rhodococcus sp., Rhodocyclus sp., Rhodomicrobium sp., Rhodopseudomonas sp., Rhodospirillum sp., Shewanella sp., Streptomyces sp., Sulfobacillus sp., Sulfolobus sp., Thiobacillus sp., Thiomicrospira sp, Thioploca sp., Thiosphaera sp., Thiothrix sp. Also chemoautotrophic microorganisms that are generally categorized as sulfur-oxidizers, hydrogen-oxidizers, iron-oxidizers, acetogens, methanogens, as well as a consortiums of microorganisms that include chemoautotrophs.

The different chemoautotrophs that can be used in the present invention may be native to a range environments including but not limited to hydrothermal vents, geothermal vents, hot springs, cold seeps, underground aquifers, salt lakes, saline formations, mines, acid mine drainage, mine tailings, oil wells, refinery wastewater, coal seams, the deep sub-surface, waste water and sewage treatment plants, geothermal power plants, sulfatara fields, soils. They may or may not be extremophiles including but not limited to thermophiles, hyperthermophiles, acidophiles, halophiles, and psychrophiles.

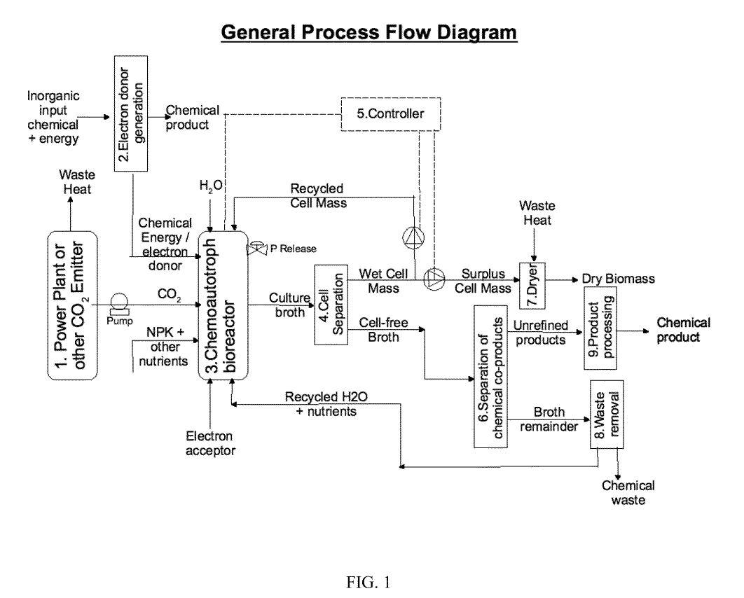

FIG. 1 illustrates the general process flow diagram for an embodiments of the present invention have a process step for the generation of electron donors suitable for supporting chemosynthesis from an energy input and raw inorganic chemical input; followed by recovery of chemical products from the electron donor generation step; delivery of generated electron donors along with electron acceptors, water, nutrients, and CO2 from a point industrial flue gas source, into chemosynthetic reaction step or steps that make use of chemoautotrophic microorganisms to capture and fix carbon dioxide, creating chemical and biomass co-products through chemosynthetic reactions; followed by process steps for the recovery of both chemical and biomass products from the process stream; and recycling of unused nutrients and process water, as well as cell mass needed to maintain the chemoautotrophic culture back into the chemosynthetic reaction steps.

Many of the reduced inorganic chemicals upon which chemoautotrophs grow (e.g. H₂, H₂S, ferrous iron, ammonium, Mn²⁺) can be readily produced using electrochemical and/or thermochemical processes known in the art of chemical engineering that can be powered by a variety carbon dioxide emission-free or low-carbon emission and/or renewable sources of power including wind, hydroelectric, nuclear, photovoltaics, or solar thermal.

Preferred embodiments of the present invention use carbon dioxide emission-free or low-carbon emission and/or renewable sources of power in the production of electron donors including but not limited to one or more of the following: photovoltaics, solar thermal, wind power, hydroelectric, nuclear, geothermal, enhanced geothermal, ocean thermal, ocean wave power, tidal power. In certain embodiments of the present invention that draw upon carbon dioxide emission-free or low-carbon emission and/or renewable sources of power in the production of electron donors, chemoautotrophs function as biocatalysts for the conversion of renewable energy into liquid hydrocarbon fuel, or high energy density organic compounds generally, with CO₂ captured from flue gases, or from the atmosphere, or ocean serving as a carbon source. These embodiments of the present invention provide renewable energy technologies with the capability of producing a transportation fuel having significantly higher energy density than if the renewable energy sources are used to produce hydrogen gas—which must be stored in relatively heavy storage systems (e.g. tanks or storage materials)—or if it is used to charge batteries which have relatively low energy density. Additionally the liquid hydrocarbon fuel product of the present invention is more compatible with the current transportation infrastructure compared to these other energy storage options. The ability of chemoautotrophs to use inorganic sources of chemical energy also enables the conversion of inorganic carbon into liquid hydrocarbon fuels using non-hydrocarbon mineralogical sources of chemical energy, i.e. reduced inorganic minerals (such as hydrogen sulfide, pyrite), which represent a largely untapped store of geochemical energy. Hence another embodiment of the present invention uses mineralogical sources of chemical energy which are pre-processed ahead of the chemosynthetic reaction steps into a form of electron donor and method of electron donor delivery that is optimal for supporting chemoautotrophic carbon fixation.

The position of the process step or steps for the generation of electron donors in the general process flow of the present invention is illustrated in FIG. 1 by the box 2. labeled “Electron Donor Generation”.

Electron donors produced in the present invention using electrochemical and/or thermochemical processes known in the art of chemical engineering and/or generated from natural sources include but are not limited to one or more of the following: ammonia; ammonium; carbon monoxide; dithionite; elemental sulfur; hydrocarbons; hydrogen; metabisulfites; nitric oxide; nitrites; sulfates such as thiosulfates including but not limited to sodium thiosulfate (Na.sub.2S.sub.2O.sub.3) or calcium thiosulfate (CaS.sub.2O.sub.3); sulfides such as hydrogen sulfide; sulfites; thionate; thionite; transition metals or their sulfides, oxides, chalcogenides, halides, hydroxides, oxyhydroxides, sulfates, or carbonates, in soluble or solid phases; as well as valence or conduction electrons in solid state electrode materials.

A preferred embodiment of the present invention uses molecular hydrogen as electron donor. Hydrogen electron donor is generated in by methods known in to art of chemical and process engineering including but not limited to more or more of the following: through electrolysis of water including but not limited to approaches using Proton Exchange Membranes (PEM), liquid electrolytes such as KOH, high-pressure electrolysis, high temperature electrolysis of steam (HTES); thermochemical splitting of water through methods including but not limited to the iron oxide cycle, cerium(IV) oxide-cerium(III) oxide cycle, zinc zinc-oxide cycle, sulfur-iodine cycle, copper-chlorine cycle, calcium-bromine-iron cycle, hybrid sulfur cycle; electrolysis of hydrogen sulfide; thermochemical splitting of hydrogen sulfide; other electrochemical or thermochemical processes known to produce hydrogen with low- or no-carbon dioxide emissions including but not limited to: carbon capture and sequestration enabled methane reforming; carbon capture and sequestration enabled coal gasification; the Kværner-process and other processes generating a carbon-black product; carbon capture and sequestration enabled gasification or pyrolysis of biomass; and the half-cell reduction of H+ to H2 accompanied by the half-cell oxidization of electron sources including but not limited to ferrous iron (Fe2+) oxidized to ferric iron (Fe3+) or the oxidation of sulfur compounds whereby the oxidized iron or sulfur can be recycled to back to a reduced state through additional chemical reaction with minerals including but not limited to metal sulfides, hydrogen sulfide, or hydrocarbons.

Certain embodiments of the present invention utilize electrochemical energy stored in solid-state valence or conduction electrons within an electrode or capacitor or related devices, alone or in combination with chemical electron donors and/or electron mediators to provide the chemoautotrophs electron donors for the chemosynthetic reactions by means of direct exposure of said electrode materials to the chemoautotrophic culturing environment.

It is preferred that embodiments of the present invention that use electrical power for the generation of electron donors, receive the electrical power from carbon dioxide emission-free or low-carbon emission and/or renewable sources of power in the production of electron donors including but not limited to one or more of the following: photovoltaics, solar thermal, wind power, hydroelectric, nuclear, geothermal, enhanced geothermal, ocean thermal, ocean wave power, tidal power.

An additional feature of the present invention regards the production, or recycling of electron donors generated from mineralogical origin including but not limited electron donors generated from reduced S and Fe containing minerals. Hence the present invention enables the use of a largely untapped source of energy—inorganic geochemical energy. There are large deposits of sulfide minerals that could be used for this purpose located in all the continents and particularly in regions of Africa, Asia, Australia, Canada, Eastern Europe, South America, and the USA. Geological sources of S and Fe such as hydrogen sulfide and pyrite, constitute a relatively inert and sizable pool of S and Fe in the respective natural cycles of sulfur and iron. Sulfides can be found in igneous rocks as well as sedimentary rocks or conglomerates. In some cases sulfides constitute the valuable part of a mineral ore, in other cases such as with coal, oil, methane, or precious metals the sulfides are considered to be impurities. In the case of fossil fuels, regulations such as Clean Air Act require the removal of sulfur impurities to prevent sulfur dioxide emissions. The use of inorganic geochemical energy facilitated by the present invention appears to be largely unprecedented, and hence the present invention represents a novel alternative energy technology.

The electron donors used in the present invention may be refined from natural mineralogical sources which include but are not limited to one or more of the following: elemental Fe.sup.0; siderite (FeCO.sub.3); magnetite (Fe.sub.3O.sub.4); pyrite or marcasite (FeS.sub.2), pyrrhotite (Fe.sub.(1-x)S (x=0 to 0.2), pentlandite (Fe,Ni).sub.9S.sub.8, violarite (Ni.sub.2FeS.sub.4), bravoite (Ni,Fe)S.sub.2, arsenopyrite (FeAsS), or other iron sulfides; realgar (AsS); orpiment (As.sub.2S.sub.3); cobaltite (CoAsS); rhodochrosite (MnCO.sub.3); chalcopyrite (CuFeS.sub.2), bornite (Cu.sub.5FeS.sub.4), covellite (CuS), tetrahedrite (Cu.sub.8Sb.sub.2S.sub.7), enargite (Cu.sub.3AsS.sub.4), tennantite (Cu.sub.12As.sub.4.S.sub.13), chalcocite (Cu.sub.2S), or other copper sulfides; sphalerite (ZnS), marmatite (ZnS), or other zinc sulfides; galena (PbS), geocronite (Pb.sub.5(Sb,As.sub.2)S.sub.8), or other lead sulfides; argentite or acanthite (Ag.sub.2S); molybdenite (MoS.sub.2); millerite (NiS), polydymite (Ni.sub.3S.sub.4) or other nickel sulfides; antimonite (Sb.sub.2S.sub.3); Ga.sub.2S.sub.3; CuSe; cooperite (PtS); laurite (RuS.sub.2); braggite (Pt,Pd,Ni)S; FeCl.sub.2.

The generation of electron donor from natural mineralogical sources includes a preprocessing step in certain embodiments of the present invention which can include but is not limited to comminuting, crushing or grinding mineral ore to increase the surface area for leaching with equipment such as a ball mill and wetting the mineral ore to make a slurry. In these embodiments of the present invention where electron donors are generated from natural mineral sources, it is preferred that particle size should be controlled so that the sulfide and/or other reducing agents present in the ore may be concentrated by methods known to the art including but not limited to: flotation methods such as dissolved air flotation or froth flotation using flotation columns or mechanical flotation cells; gravity separation; magnetic separation; heavy media separation; selective agglomeration; water separation; or fractional distillation. After the production of crushed ore or slurry, the particulate matter in the leachate or concentrate is separated by filtering (e.g. vacuum filtering), settling, or other well known techniques of solid/liquid separation, prior to introducing the electron donor containing solution to the chemoautotrophic culture environment. In addition anything toxic to the chemoautotrophs that is leached from the mineral ore is removed prior to exposing the chemoautotrophs to the leachate. The solid left after processing the mineral ore is concentrated with a filter press, disposed of, retained for further processing, or sold depending upon the mineral ore used in the particular embodiment of the invention.

The electron donors in the present invention may also be refined from pollutants or waste products including but are not limited to one or more of the following: process gas; tail gas; enhanced oil recovery vent gas; biogas; acid mine drainage; landfill leachate; landfill gas; geothermal gas; geothermal sludge or brine; metal contaminants; gangue; tailings; sulfides; disulfides; mercaptans including but not limited to methyl and dimethyl mercaptan, ethyl mercaptan; carbonyl sulfide; carbon disulfide; alkanesulfonates; dialkyl sulfides; thiosulfate; thiofurans; thiocyanates; isothiocyanates; thioureas; thiols; thiophenols; thioethers; thiophene; dibenzothiophene; tetrathionate; dithionite; thionate; dialkyl disulfides; sulfones; sulfoxides; sulfolanes; sulfonic acid; dimethylsulfoniopropionate; sulfonic esters; hydrogen sulfide; sulfate esters; organic sulfur; sulfur dioxide and all other sour gases.

In addition to mineralogical sources, electron donors are produced or recycled in certain embodiments of the present invention through chemical reactions with hydrocarbons that may be of fossil origin, but which are used in chemical reactions producing low or zero carbon dioxide gas emissions. These reactions include thermochemical and electrochemical processes. Such chemical reactions that are used in these embodiments of the present invention include but are not limited to: the thermochemical reduction of sulfate reaction or TSR and the Muller-Kuhne reaction; methane reforming-like reactions utilizing metal oxides in place of water such as but not limited to iron oxide, calcium oxide, or magnesium oxide whereby the hydrocarbon is reacted to form solid carbonate with little or no emissions of carbon dioxide gas along with hydrogen electron donor product.

The reaction formula for TSR is CaSO.sub.4+CH.sub.4→CaCO.sub.3+H.sub.2O+H.sub.2S. In this case the electron donor product that can be used by chemoautotrophic microorganisms for CO2 fixation is hydrogen sulfide (H.sub.2S) or the H.sub.2S can by further reacted electrochemically or thermochemically to produce H.sub.2 electron donor using processes known in the art of chemical engineering. The solid carbonate product (CaCO3) also formed in the TSR can be easily sequestered and applied to a number of different applications, resulting in no release of carbon dioxide into the atmosphere. There are similar reactions reducing sulfate to sulfide that involve longer chain hydrocarbons including short- and long-chain alkanes and complex aliphatic and aromatic compounds. For embodiments of the present invention using variations of the TSR is preferred that hydrocarbons sources are utilized which have little or no current economic value such as tar sand or oil shale

Examples of reactions between metal oxides and hydrocarbons to produce a hydrogen electron donor product and carbonates include but are not limited to 2CH.sub.4+Fe.sub.2O.sub.3+3H.sub.2O->2FeCO.sub.3+7H.sub.2 or CH4+CaO+2H₂O->CaCO3+4H.sub.2.

Since reactions like the TSR are exothermic, for embodiments of the present invention that utilize the TSR for electron donor generation it is preferred that heat energy released by the TSR is recovered using heat exchange methods known in the art of process engineering, to improve the efficiency of the overall process. One embodiment of the invention uses heat released by the TSR as a heat source for maintaining the proper bioreactor temperature or drying the biomass.

The generated electron donors are oxidized in the chemosynthetic reaction step or steps by electron acceptors that include but are not limited to one or more of the following: carbon dioxide, ferric iron or other transition metal ions, nitrates, nitrites, oxygen, sulfates, or holes in solid state electrode materials.

The position of the chemosynthetic reaction step or steps in the general process flow of the present invention is illustrated in FIG. 1 by the box 3. labeled “Chemoautotroph bioreactor”.

At each step in the process where chemosynthetic reactions occur one or more types of electron donor and one or more types of electron acceptor are pumped or otherwise added to the reaction vessel as either a bolus addition, or periodically, or continuously to the nutrient medium containing chemoautotrophic organisms. The chemosynthetic reaction driven by the transfer of electrons from electron donor to electron acceptor fixes inorganic carbon dioxide into organic compounds and biomass.

In certain embodiments of the present invention electron mediators may be included in the nutrient medium to facilitate the delivery of reducing equivalents from electron donors to chemoautotrophic organisms in the presence of electron acceptors and inorganic carbon in order to kinetically enhance the chemosynthetic reaction step. This aspect of the present invention is particularly applicable to embodiments of the present invention using poorly soluble electron donors such as but not limited to H2 gas or electrons in solid state electrode materials. The delivery of reducing equivalents from electron donors to the chemoautotrophs for the chemosynthetic reaction or reactions can be kinetically and/or thermodynamically enhanced in the present invention through means including but not limited to: the introduction of hydrogen storage materials into the chemoautotrophic culture environment that can double as a solid support media for microbial growth—bringing absorbed or adsorbed hydrogen electron donors into close proximity with the hydrogen-oxidizing chemoautotrophs; the introduction of electron mediators known in the art such as but not limited to cytochromes, formate, methyl-viologen, NAD+/NADH, neutral red (NR), and quinones into the chemoautotrophic culture media; the introduction of electrode materials that can double as a solid growth support media directly into the chemoautotrophic culture environment—bringing solid state electrons into close proximity with the microbes.

The culture broth used in the chemosynthetic steps of the present invention is an aqueous solution containing suitable minerals, salts, vitamins, cofactors, buffers, and other components needed for microbial growth, known to those skilled in the art [Bailey and Ollis, Biochemical Engineering Fundamentals, 2nd ed; pp 383-384 and 620-622; McGraw-Hill: New York (1986)]. These nutrients are chosen to maximize chemoautotrophic growth and promote the chemosynthetic enzymatic pathways. Alternative growth environments such as used in the arts of solid state or non-aqueous fermentation are possible. In preferred embodiments that utilize an aqueous culture broth, salt water, sea water, or other non-potable sources of water are used when tolerated by the chemoautotrophic organisms.

The chemosynthetic pathways are controlled and optimized in the present invention for the production of chemical products and/or biomass by maintaining specific growth conditions (e.g. levels of nitrogen, oxygen, phosphorous, sulfur, trace micronutrients such as inorganic ions, and if present any regulatory molecules that might not generally be considered a nutrient or energy source). Depending upon the embodiment of the invention the broth may be maintained in aerobic, microaerobic, anoxic, anaerobic, or facultative conditions depending upon the requirements of the chemoautotrophic organisms and the desired products to be created by the chemosynthetic process. A facultative environment is considered to be one having aerobic upper layers and anaerobic lower layers caused by stratification of the water column.

The source of inorganic carbon used in the chemosynthetic reaction process steps of the present invention includes but is not limited to one or more of the following: a carbon dioxide-containing gas stream that may be pure or a mixture; liquefied CO₂; dry ice; dissolved carbon dioxide, carbonate ion, or bicarbonate ion in solutions including aqueous solutions such as sea water; inorganic carbon in a solid form such as a carbonate or bicarbonate minerals. Carbon dioxide and/or other forms of inorganic carbon is introduced to the nutrient medium contained in reaction vessels either as a bolus addition or periodically or continuously at the steps in the process where chemosynthesis occurs. In preferred embodiments of the present invention, carbon dioxide containing flue gases are captured from the smoke stack at temperature, pressure, and gas composition characteristic of the untreated exhaust, and directed with minimal modification into the reaction vessels where chemosynthesis occurs in the present invention. Provided impurities harmful to chemoautotrophic organisms are not present in the flue gas, it is preferred that the modification of the flue gas upon entering the reaction vessels be limited to compression needed to pump the gas through the reactor system and heat exchange needed to lower the gas temperature to one suitable for the microorganisms.

Gases in addition to carbon dioxide that are dissolved into the culture broth of the present invention include gaseous electron donors in certain embodiments such as but not limited to hydrogen, carbon monoxide, hydrogen sulfide or other sour gases; and for aerobic embodiments of the present invention, oxygen electron acceptor, generally from air (e.g. 20.9% oxygen). The dissolution of these and other gases into solution is achieved in the present invention using a system of compressors, flowmeters, and flow valves known to one of skilled in the art of bioreactor scale microbial culturing, that feed into one of more of the following widely used systems for pumping gas into solution: sparging equipment; diffusers including but not limited to dome, tubular, disc, or doughnut geometries; coarse or fine bubble aerators; venturi equipment. In certain embodiments of the present invention surface aeration may also be performed using paddle aerators and the like. In certain embodiments of the present invention gas dissolution is enhanced by mechanical mixing with an impeller or turbine, as well as hydraulic shear devices to reduce bubble size. Following passage through the reactor system holding chemoautotrophic microorganisms which capture the carbon dioxide, the scrubbed flue gas, which is generally comprised primarily of inert gases such as nitrogen, is released into the atmosphere.

In preferred embodiments of the present invention utilizing hydrogen as electron donor, hydrogen gas is fed to the chemoautotrophic culture vessel either by bubbling it through the culture medium, or by diffusing it through a membrane that bounds the culture medium. The latter method is considered safer since hydrogen accumulating in the gas phase can create explosive conditions (the range of explosive hydrogen concentrations in air is 4 to 74.5% and is avoided in the present invention).

In aerobic embodiments of the present invention that require the pumping of air or oxygen into the culture broth in order to maintain oxygenated levels, oxygen bubbles are injected into the broth at the optimal diameter for mixing and oxygen transfer. This has been found to be 2 mm in the Environment Research Journal May/June 1999 pgs. 307-315. In certain aerobic embodiments of the present invention a process of shearing the oxygen bubbles is used to achieve this bubble diameter as described in U.S. Pat. No. 7,332,077. Bubbles should be no larger than 7.5 mm average diameter and slugging should be avoided.

Additional chemicals required for chemoautotrophic maintenance and growth as known in the art are added to the culture broth of the present invention. These chemicals may include but are not limited to: nitrogen sources such as ammonia, ammonium (e.g. ammonium chloride (NH.sub.4 Cl), ammonium sulfate ((NH.sub.4).sub.2SO.sub.4)), nitrate (e.g. potassium nitrate (KNO.sub.3)), urea or an organic nitrogen source; phosphate (e.g. disodium phosphate (Na.sub.2 HPO.sub.4), potassium phosphate (KH.sub.2 PO.sub.4), phosphoric acid (H.sub.3PO.sub.4), potassium dithiophosphate (K.sub.3PS.sub.2O.sub.2), potassium orthophosphate (K.sub.3PO.sub.4), dipotassium phosphate (K.sub.2 HPO.sub.4)); sulfate; yeast extract; chelated iron; potassium (e.g. potassium phosphate (KH.sub.2 PO.sub.4), potassium nitrate (KNO.sub.3), potassium iodide (KI), potassium bromide (KBr)); and other inorganic salts, minerals, and trace nutrients (e.g. sodium chloride (NaCl), magnesium sulfate (MgSO.sub.4 7H.sub.2O) or magnesium chloride (MgCl.sub.2), calcium chloride (CaCl.sub.2) or calcium carbonate (CaCO.sub.3), manganese sulfate (MnSO.sub.4 7H.sub.2O) or manganese chloride (MnCl.sub.2), ferric chloride (FeCl.sub.3), ferrous sulfate (FeSO.sub.4 7H.sub.2O) or ferrous chloride (FeCl.sub.2 4H.sub.2O), sodium bicarbonate (NaHCO.sub.3) or sodium carbonate (Na.sub.2CO.sub.3), zinc sulfate (ZnSO.sub.4) or zinc chloride (ZnCl.sub.2), ammonium molybdate (NH.sub.4MoO.sub.4) or sodium molybdate (Na.sub.2MoO.sub.4 2H.sub.2O), cuprous sulfate (CuSO.sub.4) or copper chloride (CuCl.sub.2 2H.sub.2O), cobalt chloride (CoCl.sub.2 6H.sub.2O), aluminum chloride (AlCl.sub.3.6H.sub.2O), lithium chloride (LiCl), boric acid (H.sub.3BO.sub.3), nickel chloride NiCl.sub.2 6H.sub.2O), tin chloride (SnCl.sub.2H.sub.2O), barium chloride (BaCl.sub.2 2H.sub.2O), copper selenate (CuSeO.sub.4 5H.sub.2O) or sodium selenite (Na.sub.2SeO.sub.3), sodium metavanadate (NaVO.sub.3), chromium salts).

The concentrations of nutrient chemicals, and particularly the electron donors and acceptors, are maintained as close as possible to their respective optimal levels for maximum chemoautotrophic growth and/or carbon uptake and fixation and/or production of organic compounds, which varies depending upon the chemoautotrophic species utilized but is known to one of skilled in the art of culturing chemoautotrophs.

Along with nutrient levels, the waste product levels, pH, temperature, salinity, dissolved oxygen and carbon dioxide, gas and liquid flow rates, agitation rate, and pressure in the chemoautotrophic culture environment are controlled in the present invention as well. The operating parameters affecting chemoautotrophic growth are monitored with sensors (e.g. dissolved oxygen probe or oxidation-reduction probe to gauge electron donor/acceptor concentrations), and controlled either manually or automatically based upon feedback from sensors through the use of equipment including but not limited to actuating valves, pumps, and agitators. The temperature of the incoming broth as well as incoming gases is regulated means such as but not limited to heat exchangers.

The dissolution of gases needed for microbial growth and metabolism, as well as the distribution of nutrients and removal of inhibitory waste products, is generally enhanced by agitation of the culture broth. Since chemoautotrophs can carry out chemosynthetic reactions throughout the volume of the reaction vessel, this gives a competitive advantage chemoautotrophic systems for carbon capture and fixation processes over rival approaches using photosynthetic organisms that are surface area limited due to the light requirements of photosynthesis. Agitation helps support this advantage by distributing the chemoautotrophs, nutrients, optimal growth environment, and CO₂ as widely and evenly as possible throughout the reactor volume so that the reactor volume in which chemosynthetic reactions occur at an optimal rate is maximized.

Agitation of the culture broth in the present invention is accomplished by equipment including but not limited to: recirculation of broth from the bottom of the container to the top via a recirculation conduit; sparging with carbon dioxide plus in certain embodiments electron donor gas (e.g. H₂ or H₂S), and for aerobic embodiments of the present invention oxygen or air as well; a mechanical mixer such as but not limited to an impeller (100-1000 rpm) or turbine.

In certain embodiments of the present invention the chemical environment, chemoautotrophic microorganisms, electron donors, electron acceptors, oxygen, pH, and temperature levels are varied either spatially and/or temporally over a series of bioreactors in fluid communication, such that a number of different chemosynthetic reactions are carried out sequentially or in parallel.

The chemoautotrophic microorganism containing nutrient medium is removed from the chemosynthetic reactors in the present invention partially or completely, periodically or continuously, and is replaced with fresh cell-free medium to maintain the cell culture in exponential growth phase and/or replenish the depleted nutrients in the growth medium and/or remove inhibitory waste products.

The production of useful chemical products through the chemosynthetic reaction step or steps reacting electron donors and acceptors to fix carbon dioxide is a feature of the present invention. These useful chemical products, both organic and inorganic, of the present invention can include but are not limited to one or more of the following: acetic acid, other organic acids and salts of organic acids, ethanol, butanol, methane, hydrogen, hydrocarbons, sulfuric acid, sulfate salts, elemental sulfur, sulfides, nitrates, ferric iron and other transition metal ions, other salts, acids or bases. Optimizing the production of a desired chemical product of chemosynthesis is achieved in the present invention through control of the parameters in the chemoautotrophic culture environment including but not limited to: nutrient levels, waste levels, pH, temperature, salinity, dissolved oxygen and carbon dioxide, gas and liquid flow rates, agitation rate, and pressure

The high growth rate of certain chemoautotrophic species enables them to equal or even surpass the highest rates of carbon fixation, and biomass production per standing unit biomass attainable by photosynthetic microbes. Consequently the production of surplus biomass is a feature of the present invention. Surplus growth of cell mass is removed from the system to produce a biomass product, and in order to maintain an optimal microbial population and cell density in the chemoautotrophic culture for continued high carbon capture and fixation rates.

Another feature of the present invention is the vessels used to contain the chemosynthetic reaction environment in the carbon capture and fixation process. The culture vessels that can be used in the present invention to culture and grow the chemoautotrophic bacteria for carbon dioxide capture and fixation are known in the art of large scale microbial culturing. These culture vessels, which may be of natural or artificial origin, include but are not limited to: airlift reactors; biological scrubber columns; bioreactors; bubble columns; caverns; caves; cisterns; continuous stirred tank reactors; counter-current, upflow, expanded-bed reactors; digesters and in particular digester systems such as known in the prior arts of sewage and waste water treatment or bioremediation; filters including but not limited to trickling filters, rotating biological contactor filters, rotating discs, soil filters; fluidized bed reactors; gas lift fermenters; immobilized cell reactors; lagoons; membrane biofilm reactors; mine shafts; pachuca tanks; packed-bed reactors; plug-flow reactors; ponds; pools; quarries; reservoirs; static mixers; tanks; towers; trickle bed reactors; vats; wells—with the vessel base, siding, walls, lining, or top constructed out of one or more materials including but not limited to bitumen, cement, ceramics, clay, concrete, epoxy, fiberglass, glass, macadam, plastics, sand, sealant, soil, steels or other metals and their alloys, stone, tar, wood, and any combination thereof. In embodiments of the present invention where the chemoautotrophic microorganisms either require a corrosive growth environment and/or produce corrosive chemicals through the chemosynthetic metabolism corrosion resistant materials are used to line the interior of the container contacting the growth medium.

Since chemoautotrophs do not require sunlight in order to fix CO₂, they can be used in carbon capture and fixation processes that avoid many of the shortcomings found for photosynthetically based technologies. Specifically the maintenance of chemosynthesis does not require shallow, wide ponds, nor bioreactors with high surface area to volume ratios and special features like solar collectors or transparent materials. A technology using chemoautotrophs does not have the diurnal, geographical, meteorological, or seasonal constraints of photosynthetically based systems.

Preferred embodiments of the present invention will minimize material costs by using chemosynthetic vessel geometries having a low surface area to volume ratio, such as but not limited to cubic, cylindrical shapes with medium aspect ratio, ellipsoidal or “egg-shaped”, hemispherical, or spherical shapes, unless material costs are superseded by other design considerations (e.g. land footprint size). The ability to use compact reactor geometries is enabled by the absence of a light requirement for chemosynthetic reactions, in contrast to photosynthetic technologies where the surface area to volume ratio must be large to provide sufficient light exposure.

The chemoautotrophs lack of dependence on light also allows plant designs with a much smaller footprint than photosynthetic approaches allow. In situations where the plant footprint needs to be minimized due to restricted land availability, the preferred embodiment of the present invention will use a long vertical shaft bioreactor system for chemoautotrophic growth and carbon capture. A bioreactor of the long vertical shaft type is described in U.S. Pat. Nos. 4,279,754, 5,645,726, 5,650,070, and 7,332,077.

Unless superseded by other considerations, preferred embodiments of the present invention will minimize vessel surfaces across which high losses of water, nutrients, and/or heat occur, or the introduction of invasive predators into the reactor. The ability to minimize such surfaces is enabled by the lack of light requirements for chemosynthesis. Photosynthetic based technologies don't have this option since surfaces across which high losses of water, nutrients, and/or heat occur, as well as losses due to predation are generally the same surfaces across which the light energy necessary for photosynthesis is transmitted.

The culture vessels of the present invention use reactor designs known in the art of large scale microbial culture to maintain an aerobic, microaerobic, anoxic, anaerobic, or facultative environment depending upon the embodiment of the present invention. Following the prior art of sewage treatment, in certain embodiments of the present invention tanks are arranged in a sequence, with serial forward fluid communication, where certain tanks are maintained in aerobic conditions and others are maintained in anaerobic conditions, in order to perform multiple chemoautotrophic processing steps on the carbon dioxide waste stream.

In certain embodiments of the present invention the chemoautotrophic microorganisms are immobilized within their growth environment. This is accomplished using any media known in the art of microbial culturing to support colonization by chemoautotrophic microorganisms including but not limited to growing the chemoautotrophs on a matrix, mesh, or membrane made from any of a wide range of natural and synthetic materials and polymers including but not limited to one or more of the following: glass wool, clay, concrete, wood fiber, inorganic oxides such as ZrO.sub.2, Sb.sub.2 O.sub.3, or Al.sub.2 O.sub.3, the organic polymer polysulfone, or open-pore polyurethane foam having high specific surface area. The chemoautotrophic microorganisms in the present invention may also be grown on the surfaces of unattached objects distributed throughout the growth container as are known in the art of microbial culturing that include but are not limited to one or more of the following: beads; sand; silicates; sepiolite; glass; ceramics; small diameter plastic discs, spheres, tubes, particles, or other shapes known in the art; shredded coconut hulls; ground corn cobs; activated charcoal; granulated coal; crushed coral; sponge balls; suspended media; bits of small diameter rubber (elastomeric) polyethylene tubing; hanging strings of porous fabric, Berl saddles, Raschig rings.

Inoculation of the chemoautotrophic culture into the culture vessel is performed by methods including but not limited to transfer of culture from an existing chemoautotrophic culture inhabiting another carbon capture and fixation system of the present invention, or incubation from a seed stock raised in an incubator. The seed stock of chemoautotrophic strains is transported and stored in forms including but not limited to a powder, liquid, frozen, or freeze-dried form as well as any other suitable form, which may be readily recognized by one skilled in the art. When establishing a culture in a very large reactor it is preferable to grow and establish cultures in progressively larger intermediate scale containers prior to inoculation of the full scale vessel.

The position of the process step or steps for the separation of cell mass from the process stream in the general process flow of the present invention is illustrated in FIG. 1 by the box 4. labeled “Cell Separation”.

Separation of cell mass from liquid suspension in the present invention is performed by methods known in the art of microbial culturing [Examples of cell mass harvesting techniques are given in International Patent Application No. WO08/00558, published Jan. 8, 1998; U.S. Pat. No. 5,807,722; U.S. Pat. No. 5,593,886 and U.S. Pat. No. 5,821,111.] including but not limited to one or more of the following: centrifugation; flocculation; flotation; filtration using a membranous, hollow fiber, spiral wound, or ceramic filter system; vacuum filtration; tangential flow filtration; clarification; settling; hydrocyclone. In embodiments where the cell mass is immobilized on a matrix it is harvested by methods including but not limited to gravity sedimentation or filtration, and separated from the growth substrate by liquid shear forces.

In the present invention, if an excess of cell mass has been removed from the culture, it is recycled back into the cell culture as indicated by the process arrow labeled “Recycled Cell Mass” in FIG. 1., along with fresh broth such that sufficient biomass is retained in the chemosynthetic reaction step or steps for continued optimal inorganic carbon uptake and growth or metabolic rate. The cell mass recovered by the harvesting system is recycled back into the culture vessel using an airlift or geyser pump. It is preferred that the cell mass recycled back into the culture vessel has not been exposed to flocculating agents, unless those agents are non-toxic to the chemoautotrophs.

In preferred embodiments of the present invention the chemoautotrophic system is maintained, using continuous influx and removal of nutrient medium and/or biomass, in steady state where the cell population and environmental parameters (e.g. cell density, chemical concentrations) are targeted at a constant optimal level over time. Cell densities are monitored in the present invention either by direct sampling, by a correlation of optical density to cell density, or with a particle size analyzer. The hydraulic and biomass retention times are decoupled so as to allow independent control of both the broth chemistry and the cell density. Dilution rates are kept high enough so that the hydraulic retention time is relatively low compared to the biomass retention time, resulting in a highly replenished broth for cell growth. Dilution rates are set at an optimal trade-off between culture broth replenishment, and increased process costs from pumping, increased inputs, and other demands that rise with dilution rates.

To assist in the processing of the biomass product into biofuels or other useful products, the surplus microbial cells in certain embodiments of the invention are broken open following the cell recycling step using methods including but not limited to ball milling, cavitation pressure, sonication, or mechanical shearing.

The harvested biomass in the present invention is dried in the process step or steps of box 7. labeled “Dryer” in the general process flow of the present invention illustrated in FIG. 1.

Surplus biomass drying is performed in the present invention using technologies including but not limited to centrifugation, drum drying, evaporation, freeze drying, heating, spray drying, vacuum drying, vacuum filtration. Heat waste from the industrial source of flue gas is preferably used in drying the biomass. In addition the chemosynthetic oxidation of electron donors is exothermic and generally produces waste heat. In preferred embodiments of the present invention waste heat will be used in drying the biomass.

In certain embodiments of the invention the biomass is further processed following drying to aid the production of biofuels or other useful chemicals through the separation of the lipid content or other targeted biochemicals from the chemoautotrophic biomass.

The separation of the lipids is performed by using nonpolar solvents to extract the lipids such as, but not limited to, hexane, cyclohexane, ethyl ether, alcohol (isopropanol, ethanol, etc.), tributyl phosphate, supercritical carbon dioxide, trioctylphosphine oxide, secondary and tertiary amines, or propane. Other useful biochemicals can be extracted using solvents including but not limited to: chloroform, acetone, ethyl acetate, and tetrachloroethylene.

The broth left over following the removal of cell mass is pumped to a system for removal of the products of chemosynthesis and/or spent nutrients which are recycled or recovered to the extent possible, or else disposed of

The position of the process step or steps for the recovery of chemical products from the process stream in the general process flow of the present invention is illustrated in FIG. 1 by the box 6. labeled “Separation of chemical products”.

Recovery and/or recycling of chemosynthetic chemical products and/or spent nutrients from the aqueous broth solution is accomplished in the present invention using equipment and techniques known in the art of process engineering, and targeted towards the chemical products of particular embodiments of the present invention, including but not limited to: solvent extraction; water extraction; distillation; fractional distillation; cementation; chemical precipitation; alkaline solution absorption; absorption or adsorption on activated carbon, ion-exchange resin or molecular sieve; modification of the solution pH and/or oxidation-reduction potential, evaporators, fractional crystallizers, solid/liquid separators, nanofiltration, and all combinations thereof.

Following the recovery of useful or valuable products from the process stream the removal of the waste products is performed as indicated by the box 8. labeled “Waste removal” in FIG. 1. The remaining broth is returned to the culture vessel along with replacement water and nutrients.

In embodiments of the present invention involving chemoautotrophic oxidization of electron donors extracted from the mineral ore, there will generally remain a solution of oxidized metal cations following the chemosynthetic reaction steps. A solution rich in dissolved metal cations can also result from a particularly dirty flue gas input to the process such as from a coal fired plant. In these embodiment of the present invention the process stream is stripped of metal cations by methods including but not limited to: cementation on scrap iron, steel wool, copper or zinc dust; chemical precipitation as a sulfide or hydroxide precipitate; electrowinning to plate a specific metal; absorption on activated carbon or an ion-exchange resin, modification of the solution pH and/or oxidation-reduction potential, solvent extraction. In certain embodiments of the present invention the recovered metals can be sold for an additional stream of revenue. Metals that may be recovered certain embodiments of the present invention from the mineral source of electron donors depending upon the source of the mineral may include but are not limited to one or more of the following base or precious metals: cobalt (Co), copper (Cu), gold (Au), iridium (Ir), iron (Fe), lead (Pb), manganese (Mn), osmium (Rh), platinum (Pt), palladium (Pd), rhodium (Rh), ruthenium (Ru), silver (Ag), uranium (U), zinc (Zn).

Chemicals that are used in processes for the recovery of chemical products, the recycling of nutrients and water, and the removal of waste, are preferred to have low toxicity for humans, and if exposed to the process stream that is recycled back into the growth container, low toxicity for the chemoautotrophs being used.

In certain embodiments of the present invention the chemoautotrophs used create an acid product through chemosynthesis. An example is aerobic sulfur-oxidizing chemoautotrophs which produce sulfuric acid through their chemosynthetic reaction. Preferably as much sulfuric acid product as possible is recovered from the process stream in embodiments using these microorganisms. However it may be necessary to neutralize the remainder in the broth before it is either recycled back into to the growth container or discharged into the environment. A neutralization step is performed in these embodiments prior to recycling the broth back into the culture vessel in order to maintain the pH within an optimal range for microbial maintenance and growth. A neutralization step is also performed in these embodiments when discharging into the environment to keep the pH within a safe range. Neutralization of acid in the broth can be accomplished by the addition of bases including but not limited to: limestone, lime, sodium hydroxide, ammonia, caustic potash, magnesium oxide, iron oxide. It is preferred that the base is produced from a carbon dioxide emission-free source such as naturally occurring basic minerals including but not limited to calcium oxide, magnesium oxide, iron oxide, iron ore, olivine containing a metal oxide, serpentine containing a metal oxide, ultramafic deposits containing metal oxides, and underground basic saline aquifers. In the neutralization of sulfuric acid, use of lime or limestone will precipitate calcium sulfate. The precipitate can then be removed by vacuum filtration or some other solid/liquid separation method known in the art of process engineering and solid gypsum cake recovered. If limestone is used for neutralization, then carbon dioxide will be released which is either directed back into the growth container for uptake by the chemoautotrophs, or sequestered in some other way, rather than released into the atmosphere, in preferred embodiments. If neutralized sulfates are returned to the growth container care is taken that they do not reach inhibitory concentrations. The counter ion to the sulfate which is determined by base used in neutralization can strongly influence the level of sulfate that can be tolerated by the chemoautotrophs as discussed in U.S. Pat. No. 4,859,588.

In addition to carbon dioxide captured through the chemosynthetic fixation of carbon, additional carbon dioxide can be captured and converted to carbonates or biominerals through the catalytic action of chemoautotrophic microorganisms in certain embodiments of the present invention. For embodiments of the invention that augment the carbon captured through chemosynthesis with biocatalyzed mineral carbon sequestration, the use of chemoautotrophic microorganisms capable of withstanding a high pH solution where carbon dioxide is thermodynamically favored to precipitate as carbonate is preferred. Any carbonate or biomineral precipitate produced will be removed periodically or continuously from the system using solid/liquid separation techniques known in the art of process engineering.

An additional feature of the present invention relates to the uses of chemical products generated through the chemosynthetic carbon capture and fixation process. The chemical products of the present invention can be applied to uses including but not limited to one or more of the following: as biofuel; as feedstock for the production of biofuels; in the production of fertilizers; as a leaching agent for the chemical extraction of metals in mining or bioremediation; as chemicals reagents in industrial or mining processes.

An additional feature of the present invention relates to the uses of biochemicals or biomass produced through the chemosynthetic process step or steps of the present invention. Uses of the biomass product include but are not limited to: as a biomass fuel for combustion in particular as a fuel to be co-fired with fossil fuels such as coal in pulverized coal powered generation units; as a carbon source for large scale fermentations to produce various chemicals including but not limited to commercial enzymes, antibiotics, amino acids, vitamins, bioplastics, glycerol, or 1,3-propanediol; as a nutrient source for the growth of other microbes or organisms; as feed for animals including but not limited to cattle, sheep, chickens, pigs, or fish; as feed stock for alcohol or other biofuel fermentation and/or gasification and liquefaction processes including but not limited to direct liquefaction, Fisher Tropsch processes, methanol synthesis, pyrolysis, or microbial syngas conversions, for the production of liquid fuel; as feed stock for methane or biogas production; as fertilizer; as raw material for manufacturing or chemical processes such as but not limited to the production of biodegradable/biocompatible plastics; as sources of pharmaceutical, medicinal or nutritional substances; soil additives and soil stabilizers.

An additional feature of the present invention relates to using carbohydrate and/or sugar content of the biomass to provide substrate for fermentation reactions by ethanol-producing microorganisms including but not limited to Saccharomyces sp., Candida sp. and Brettanomyces sp. The biochemical feedstock provided by chemoautotrophic microorganisms for fermentation is a combination of sugars, carbohydrates, and/or starches that have been separated from the cell mass using any of a number of different methods known in the arts of biorefining.

For embodiments of the present invention utilizing Sulfur oxidizing chemoautotrophs which generate sulfuric acid as a co-product of the chemosynthetic metabolism, preferred embodiments utilize some of the sulfuric acid co-product in hydrolyzing the carbohydrates and/or starches extracted from the chemoautotrophic cell mass into simpler sugars that are suitable for fermentation. Ethanol produced from fermentation of the simple sugars is volatile and miscible with aqueous solutions, and is generally separated by a distillation process. The large scale production of cheap carbohydrates enabled by the present invention is useful to the fermentation industry where the cost of carbohydrates represents a major proportion of the overall cost of fermentation [Crueger and Crueger, Biotechnology: A Textbook of Industrial Microbiology, Sinauer Associates: Sunderland, Mass., pp 124-174 (1990); Atkinson and Mavituna, Biochemical Engineering and Biotechnology Handbook, 2.sup.nd ed.; Stockton Press: New York, pp 243-364 (1991)].

An additional feature of the present invention relates to the optimization of chemoautotrophic organisms for carbon dioxide capture, carbon fixation into organic compounds, and the production of other valuable chemical co-products. This optimization can occur through methods known in the art of artificial breeding including but not limited to accelerated mutagenesis (e.g. using ultraviolet light or chemical treatments), genetic engineering or modification, hybridization, synthetic biology or traditional selective breeding. For embodiments of the present invention utilizing a consortium of chemoautotrophs the community can be enriched with desirable organisms using methods known in the art of microbiology through growth in the presence of target electron donors, acceptors, and environmental conditions.

An additional feature of the present invention relates to modifying biochemical pathways in chemoautotrophs for the production of targeted organic compounds. This modification can be either be accomplished by manipulating the growth environment, or through methods known in the art of artificial breeding including but not limited to accelerated mutagenesis (e.g. using ultraviolet light or chemical treatments), genetic engineering or modification, hybridization, synthetic biology or traditional selective breeding. The organic compounds produced through the modification include but are not limited to: biofuels including but not limited to biodiesel or renewable diesel, ethanol, gasoline, long chain hydrocarbons, methane and pseudovegetable oil produced from biological reactions in vivo; or organic compounds or biomass optimized as a feedstock for biofuel and/or liquid fuel production through chemical processes. These forms of fuel can be used as renewable/alternate sources of energy with low greenhouse gas emissions.

In order to give specific examples of the overall biological and chemical process for using chemoautotrophic microorganisms to capture CO.sub.2 and produce biomass and other useful co-products, a number of process flow diagrams describing various embodiments of the present invention are now provided and described. These specific examples should not be construed as limiting the present invention in any way and are provided for the sole purpose of illustration.

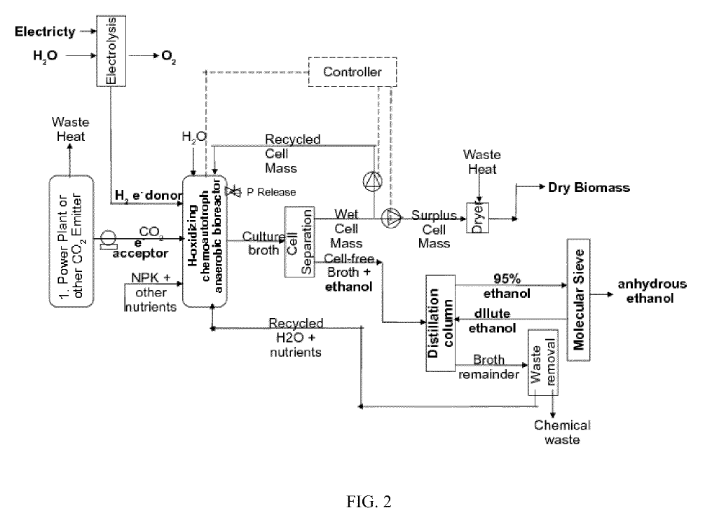

FIG. 2 is process flow diagram for the preferred embodiment of the present invention for the capture of CO.sub.2 by hydrogen oxidizing chemoautotrophs and production of ethanol. A carbon dioxide rich flue gas is captured from an emission source such as a power plant, refinery, or cement producer. The flue gas is then compressed and pumped into cylindrical anaerobic digesters containing one or more hydrogen oxidizing acetogenic chemoautotrophs such as but not limited to Acetoanaerobium noterae, Acetobacterium woodii, Acetogenium kivui, Butyribacterium methylotrophicum, Butyribacterium rettgeri, Clostridium aceticum, Clostridium acetobutylicum, Clostridium acidi-urici, Clostridium autoethanogenum, Clostridium carboxidivorans, Clostridium formicoaceticum, Clostridium kluyveri, Clostridium ljungdahlii, Clostridium thermoaceticum, Clostridium thermoautotrophicum, Clostridium thermohydrosulfuricum, Clostridium thermosaccharolyticum, Clostridium thermocellum, Eubacterium limosum, Peptostreptococcus productus. Hydrogen electron donor is added continuously to the growth broth along with other nutrients required for chemoautotrophic growth and maintenance that are pumped into the digester. It is preferred that the hydrogen source is a carbon dioxide emission-free process. This could be electrolytic or thermochemical processes powered by energy technologies including but not limited to photovoltaics, solar thermal, wind power, hydroelectric, nuclear, geothermal, enhanced geothermal, ocean thermal, ocean wave power, tidal power. Carbon dioxide serves as an electron acceptor in the chemosynthetic reaction. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the ethanol product is distilled put through a molecular sieve to produce anhydrous ethanol using standard techniques known in the art of distillation. The broth left over after distillation is then subjected to any necessary additional waste removal treatments which depends on the source of flue gas. The remaining water and nutrients are then pumped back into the digesters.

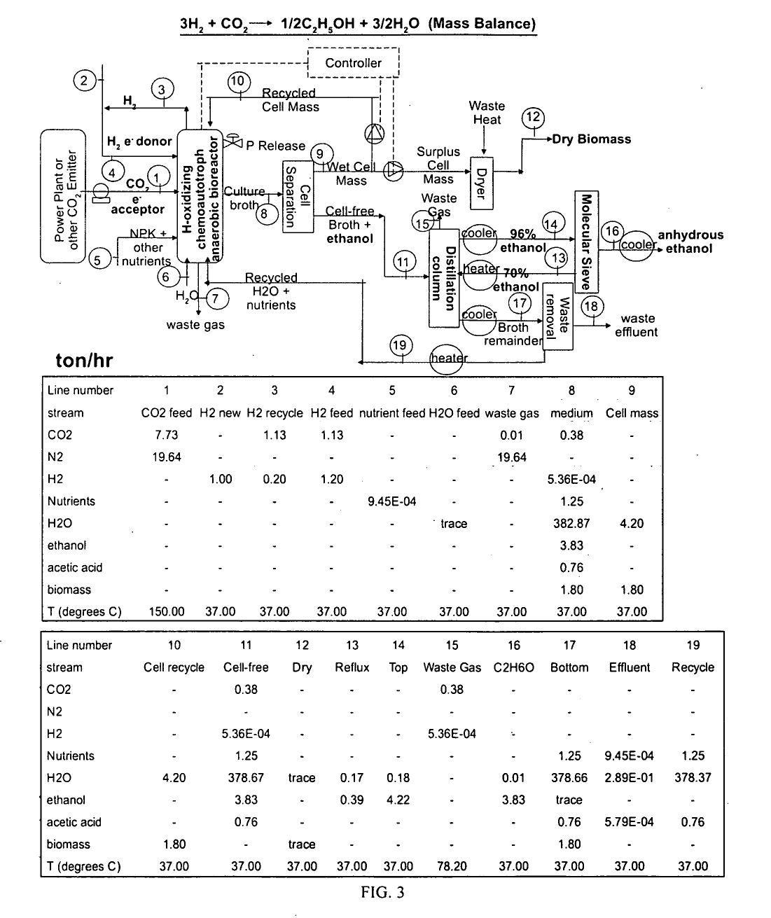

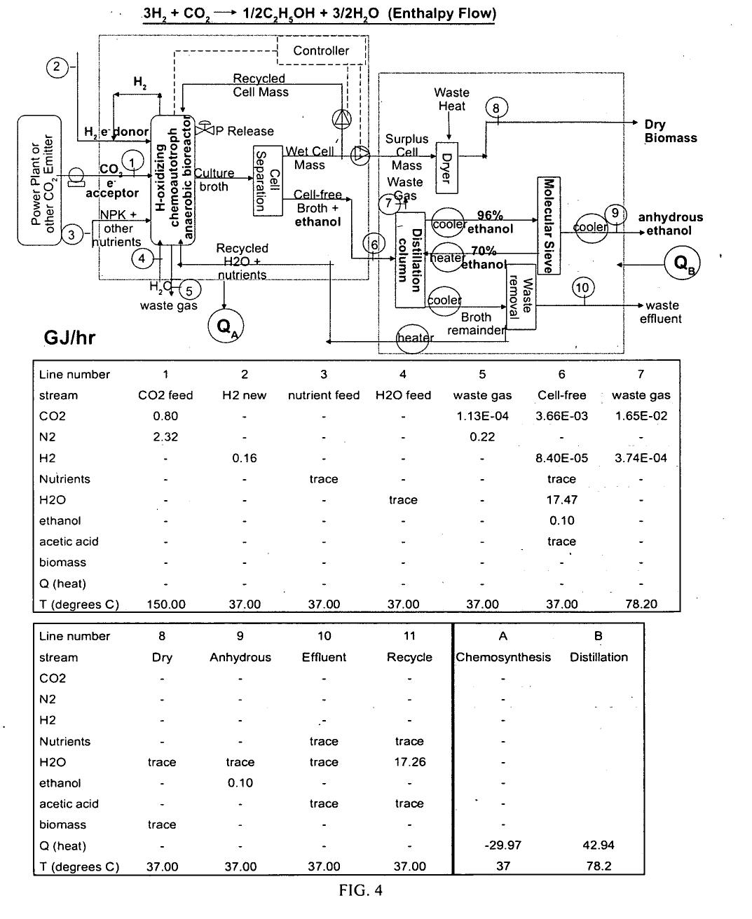

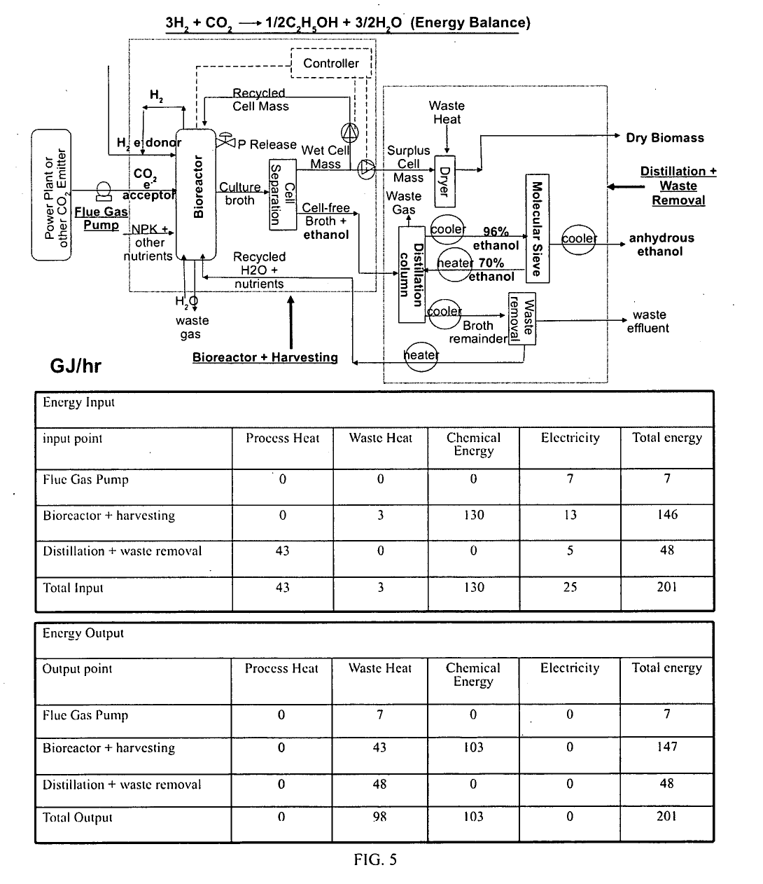

A process model is given in FIGS. 3, 4 and 5 for the preferred embodiment of the present invention using hydrogen electron donors. The mass balance, enthalpy flow, energy balance, and plant economics have been calculated for this [Sinnott, 2005] preferred embodiment for the present invention. The model was developed using established results in the scientific literature for the H.sub.2 oxidizing acetogens and for the process steps known from the art of chemical engineering.

The mass balance indicates that 1 ton of ethanol will be produced for every 2 tons of CO₂ pumped into the system. This amounts to over 150 gallons of ethanol produced per ton of CO₂ intake. The energy balance indicates that for every GJ of H.sub.2 chemical energy input there is 0.8 GJ of ethanol chemical energy out, i.e. the chemical conversion is expected to be around 80% efficient. Overall efficiency of ethanol production from H₂ and CO₂ including electric power and process heat is predicted with the model to be about 50%.

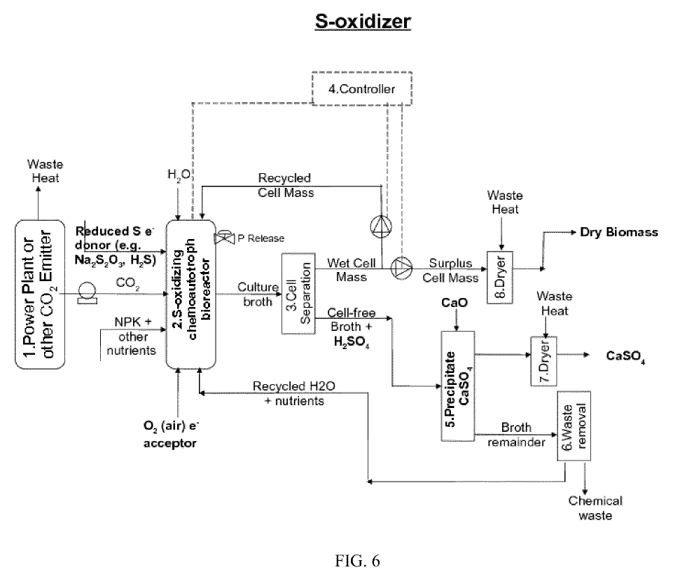

FIG. 6 is process flow diagram for the capture of CO.sub.2 by sulfur oxidizing chemoautotrophs and production of biomass and gypsum. A carbon dioxide rich flue gas is captured from an emission source such as a power plant, refinery, or cement producer. The flue gas is then compressed and pumped into cylindrical aerobic digesters containing one or more sulfur oxidizing chemoautotrophs such as but not limited to Thiomicrospira crunogena, Thiomicrospira strain MA-3, Thiomicrospira thermophile, Thiobacillus hydrothermalis, Thiomicrospira sp. strain CVO, Thiobacillus neapolitanus, Arcobacter sp. strain FWKO B. One or more electron donors such as but not limited to thiosulfate, hydrogen sulfide, or sulfur are added continuously to the growth broth along with other nutrients required for chemoautotrophic growth and air is pumped into the digester to provide oxygen as an electron acceptor. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the sulfuric acid produced by the chemosynthetic metabolism is neutralized with lime, precipitating out gypsum (CaSO.sub.4). It is preferred that the lime is produced by a carbon dioxide emission-free process rather than through the heating of limestone. Such carbon dioxide emission-free processes include the recovery of natural sources of basic minerals including but not limited to minerals containing a metal oxide, serpentine containing a metal oxide, ultramafic deposits containing metal oxides, and underground basic saline aquifers. Alternative bases may be used for neutralization in this process including but not limited to magnesium oxide, iron oxide, or some other metal oxide. The gypsum is removed by solid-liquid separation techniques and pumped to dryers. The final product is dried gypsum. The broth left over after the sulfate is precipitated out is then subjected to any necessary additional waste removal treatments which depends on the source of flue gas. The remaining water and nutrients are then pumped back into the digesters.

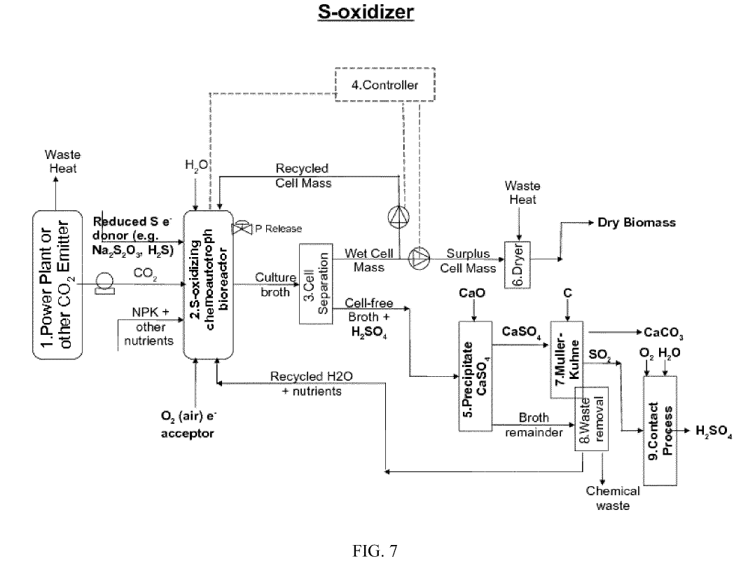

FIG. 7 is process flow diagram for the capture of CO.sub.2 by sulfur oxidizing chemoautotrophs and production of biomass and sulfuric acid and calcium carbonate via the Muller-Kuhne reaction. A carbon dioxide rich flue gas is captured from an emission source such as a power plant, refinery, or cement producer. The flue gas is then compressed and pumped into cylindrical aerobic digesters containing one or more sulfur oxidizing chemoautotrophs such as but not limited to Thiomicrospira crunogena, Thiomicrospira strain MA-3, Thiomicrospira thermophile, Thiobacillus hydrothermalis, Thiomicrospira sp. strain CVO, Thiobacillus neapolitanus, Arcobacter sp. strain FWKO B. One or more electron donors such as but not limited to thiosulfate, hydrogen sulfide, or sulfur are added continuously to the growth broth along with other nutrients required for chemoautotrophic growth and air is pumped into the digester to provide oxygen as an electron acceptor. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the sulfuric acid produced by the chemosynthetic metabolism is neutralized with lime (CaO), precipitating out gypsum (CaSO.sub.4). It is preferred that the lime is produced by a carbon dioxide emission-free process rather than through the heating of limestone. Such carbon dioxide emission-free processes include the recovery of natural sources of basic minerals including but not limited to minerals containing a metal oxide, iron ore, serpentine containing a metal oxide, ultramafic deposits containing metal oxides, and underground basic saline aquifers. Alternative bases may be used for neutralization in this process including but not limited to magnesium oxide, iron oxide, or some other metal oxide. The gypsum is removed by solid-liquid separation techniques and pumped to kilns where the Muller-Kuhne process is carried out with the addition of coal. The net reaction for the Muller-Kuhne process is as follows 2C+4CaSO₄→2CaO+2CaCO₃+4SO₂. The produced CaCO3 is collected and the CaO is recycled for further neutralization. The SO.sub.2 gas produced is directed to a reactor for the contact process where sulfuric acid is produced. The broth left over after the sulfate is precipitated out is then subjected to any necessary additional waste removal treatments which depends on the source of flue gas. The remaining water and nutrients are then pumped back into the digesters.

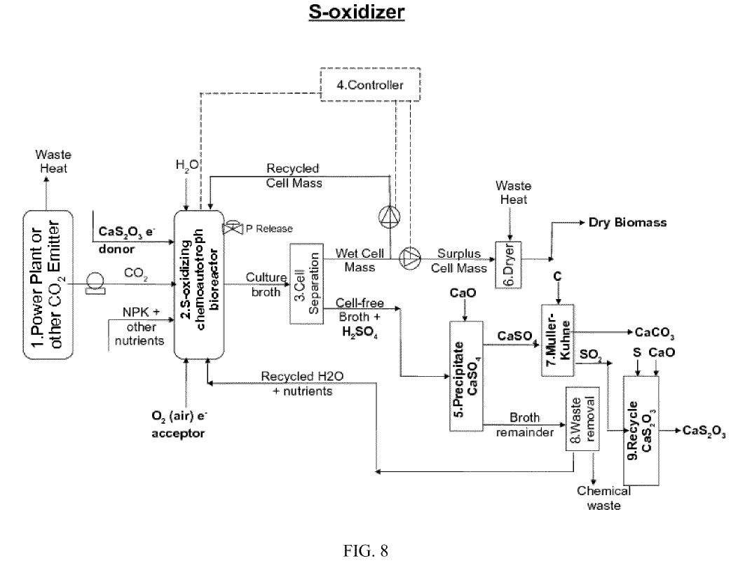

FIG. 8 is a process flow diagram for the capture of CO.sub.2 by sulfur oxidizing chemoautotrophs and production of biomass and calcium carbonate and recycling of thiosulfate electron donor via the Muller-Kuhne reaction. A carbon dioxide rich flue gas is captured from an emission source such as a power plant, refinery, or cement producer. The flue gas is then compressed and pumped into cylindrical aerobic digesters containing one or more sulfur oxidizing chemoautotrophs such as but not limited to Thiomicrospira crunogena, Thiomicrospira strain MA-3, Thiomicrospira thermophile, Thiobacillus hydrothermalis, Thiomicrospira sp. strain CVO, Thiobacillus neapolitanus, Arcobacter sp. strain FWKO B. Calcium thiosulfate is the electron donor added continuously to the growth broth along with other nutrients required for chemoautotrophic growth and air is pumped into the digester to provide oxygen as an electron acceptor. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the sulfuric acid produced by the chemosynthetic metabolism is neutralized with lime (CaO), precipitating out gypsum (CaSO.sub.4). It is preferred that the lime is produced by a carbon dioxide emission-free process rather than through the heating of limestone. Such carbon dioxide emission-free processes include the recovery of natural sources of basic minerals including but not limited to minerals containing a metal oxide, serpentine containing a metal oxide, ultramafic deposits containing metal oxides, and underground basic saline aquifers. Alternative bases may be used for neutralization in this process including but not limited to magnesium oxide, iron oxide, or some other metal oxide. The gypsum is removed by solid-liquid separation techniques and pumped to kilns where the Muller-Kuhne process is carried out with the addition of coal. The net reaction for the Muller-Kuhne process is as follows 2C+4CaSO₄→2CaO+2CaCO₃+4SO₂. The produced CaCO.sub.3 is collected and the CaO is recycled for further reaction. The SO.sub.2 gas produced is directed to a reactor where it is reacted with CaO or some other metal oxide such as iron oxide, and sulfur to recycle the thiosulfate (calcium thiosulfate if CaO is used). The broth left over after the sulfate is precipitated out is then subjected to any necessary additional waste removal treatments which depends on the source of flue gas. The remaining water and nutrients are then pumped back into the digesters.

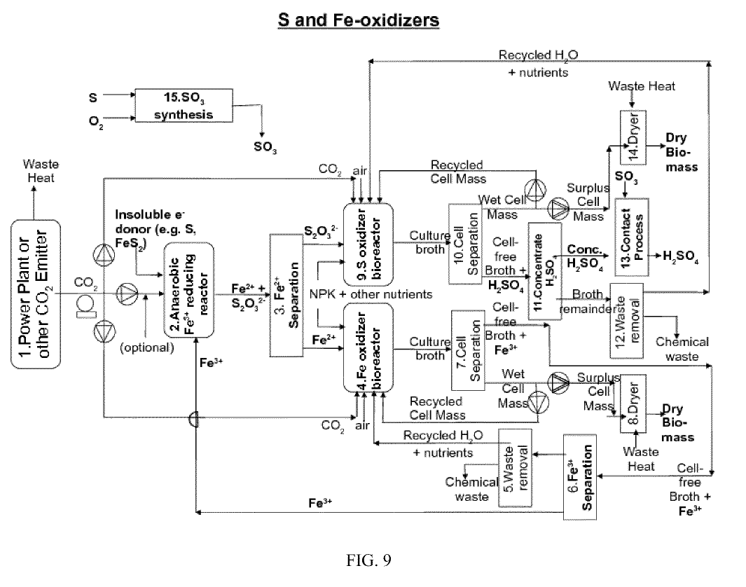

FIG. 9 is process flow diagram for the capture of CO.sub.2 by sulfur and iron oxidizing chemoautotrophs and production of biomass and sulfuric acid using an insoluble source of electron donors. A carbon dioxide rich flue gas is captured from an emission source such as a power plant, refinery, or cement producer. The flue gas is then compressed and pumped into one set of cylindrical aerobic digesters containing one or more sulfur oxidizing chemoautotrophs such as but not limited to Thiomicrospira crunogena, Thiomicrospira strain MA-3, Thiomicrospira thermophile, Thiobacillus hydrothermalis, Thiomicrospira sp. strain CVO, Thiobacillus neapolitanus, Arcobacter sp. strain FWKO B, and another set of cylindrical aerobic digesters containing one or more iron oxidizing chemoautotrophs such as but not limited to Leptospirillum ferrooxidans or Thiobacillus ferrooxidans. One or more insoluble sources of electron donors such as but not limited to elemental sulfur, pyrite, or other metal sulfides are sent to a anaerobic reactor for reaction with a ferric iron solution. Optionally chemoautotrophs such as but not limited to Thiobacillus ferrooxidans and Sulfolobus sp. can be present in this reactor to help biocatalyze the attack of the insoluble electron donor source with ferric iron. A leachate of ferrous iron and thiosulfate flow out of the reactor. The ferrous iron is separated out of the process stream by precipitation. The thiosulfate solution is then flowed into the S-oxidizer digesters and the ferrous iron is pumped into the Fe-oxidizer digesters as the electron donor for each type of chemoautotroph respectively. Air and other nutrients required for chemoautotrophic growth are also pumped into the digesters. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. In the S-oxidizer process stream the cell-free broth which has passed through the cell mass removing filters is directed to sulfuric acid recovery systems such employed in the refinery or distillery industries where the sulfuric acid product of chemosynthetic metabolism is concentrated. This sulfuric acid concentrate is then concentrated further using the contact process to give a concentrated sulfuric acid product. The broth left over after the sulfate and sulfuric acid have been removed is then subjected to any necessary additional waste removal treatments which depends on the source of flue gas. In the Fe-oxidizer process stream the cell-free broth which has passed through the cell mass removing filters is then stripped of ferric iron by precipitation. This ferric iron is then sent back for further reaction with the insoluble source of electron donors (e.g. S, FeS.sub.2). The remaining water and nutrients in both process streams are then pumped back into their respective digesters.

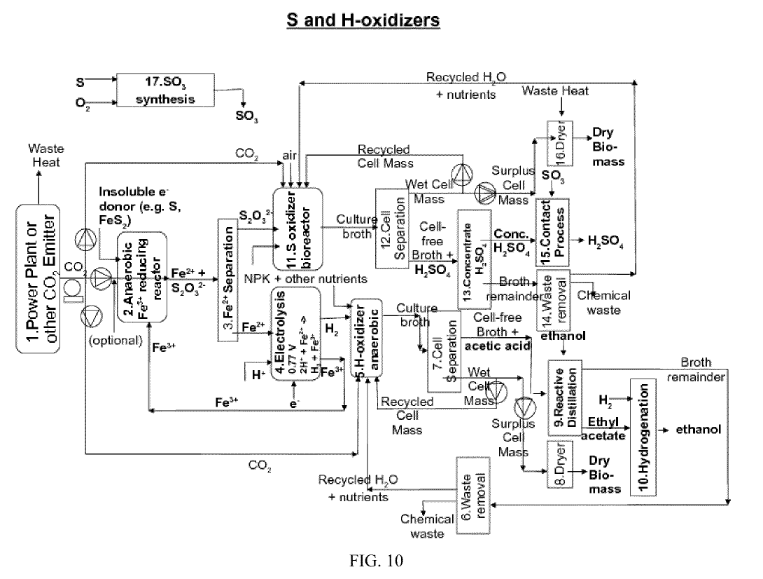

FIG. 10 is a process flow diagram for the capture of CO.sub.2 by sulfur and hydrogen oxidizing chemoautotrophs and production of biomass, sulfuric acid, and ethanol using an insoluble source of electron donors. A carbon dioxide rich flue gas is captured from an emission source such as a power plant, refinery, or cement producer. The flue gas is then compressed and pumped into one set of cylindrical aerobic digesters containing one or more sulfur oxidizing chemoautotrophs such as but not limited to Thiomicrospira crunogena, Thiomicrospira strain MA-3, Thiomicrospira thermophile, Thiobacillus hydrothermalis, Thiomicrospira sp. strain CVO, Thiobacillus neapolitanus, Arcobacter sp. strain FWKO B, and another set of cylindrical anaerobic digesters containing one or more hydrogen oxidizing acetogenic chemoautotrophs such as but not limited to Acetoanaerobium noterae, Acetobacterium woodii, Acetogenium kivui, Butyribacterium methylotrophicum, Butyribacterium rettgeri, Clostridium aceticum, Clostridium acetobutylicum, Clostridium acidi-urici, Clostridium autoethanogenum, Clostridium carboxidivorans, Clostridium formicoaceticum, Clostridium kluyveri, Clostridium ljungdahlii, Clostridium thermoaceticum, Clostridium thermoautotrophicum, Clostridium thermohydrosulfuricum, Clostridium thermosaccharolyticum, Clostridium thermocellum, Eubacterium limosum, Peptostreptococcus productus. One or more insoluble sources of electron donors such as but not limited to elemental sulfur, pyrite, or other metal sulfides are sent to an anaerobic reactor for reaction with a ferric iron solution. Optionally chemoautotrophs such as but not limited to Thiobacillus ferrooxidans and Sulfolobus sp. can be present in this reactor to help biocatalyze the attack of the insoluble electron donor source with ferric iron. A leachate of ferrous iron and thiosulfate flow out of the reactor. The ferrous iron is separated out of the process stream by precipitation. The thiosulfate solution is then flowed into the S-oxidizer digesters as an electron donor and the ferrous iron is pumped into an anaerobic electrolysis reactor. In the electrolysis reactor hydrogen gas is formed by the electrochemical reaction 2H.sup.++Fe.sup.2+→H.sub.2+Fe.sup.3+. The open cell voltage for this reaction is 0.77 V which is substantially lower than the open cell voltage for the electrolysis of water (1.23 V). Furthermore the kinetics of the oxidation of ferrous iron to ferric iron is much simpler than that for the reduction of oxygen in water to oxygen gas, hence the overvoltage for the iron reaction is lower. These factors combined provides an energy savings for the production of hydrogen gas by using ferrous iron compared to electrolysis of water. The hydrogen produced is fed into the H-oxidizer digesters as the electron donor. The other nutrients required for chemoautotrophic growth are also pumped into the digesters. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. In the S-oxidizer process stream the cell-free broth which has passed through the cell mass removing filters is directed to sulfuric acid recovery systems such as employed in the refinery and distillation industries where the sulfuric acid product of chemosynthetic metabolism is concentrated. This sulfuric acid concentrate is then concentrated further using the contact process to give a concentrated sulfuric acid product. The broth left over after the sulfate and sulfuric acid have been removed is then subjected to any necessary additional waste removal treatments which depends on the source of flue gas. In the H-oxidizer process stream the cell-free broth which has passed through the cell mass removing filters is directed to vessels where the acetic acid produced is reacted with ethanol to produce ethyl acetate which is removed from solution by reactive distillation. The ethyl acetate is converted to ethanol by hydrogenation. Half of the ethanol is recycled for further reaction in the reactive distillation process. The other half is put through a molecular sieve which separates anhydrous ethanol by adsorption from dilute ethanol. The anhydrous ethanol is then collected and the dilute ethanol is returned for further reaction in the reactive distillation step. The broth left over after the acetic acid is reactively distilled out is then subjected to any necessary additional waste removal treatments which depends on the source of flue gas. The remaining water and nutrients in both process streams are then pumped back into their respective digesters.

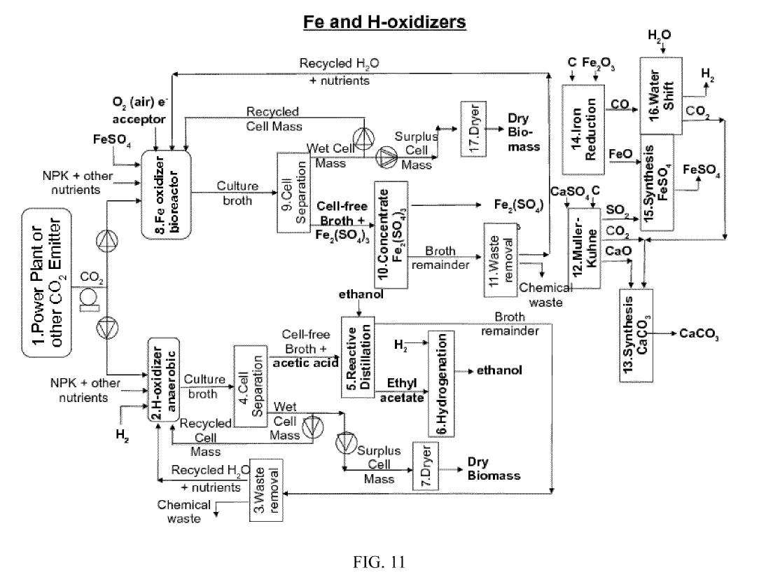

FIG. 11 is process flow diagram for the capture of CO.sub.2 by iron and hydrogen oxidizing chemoautotrophs and production of biomass, ferric sulfate, calcium carbonate and ethanol using coal or another hydrocarbon as the energy input for the production of electron donors without the release of gaseous CO.sub.2. A carbon dioxide rich flue gas is captured from an emission source such as a power plant, refinery, or cement producer. The flue gas is then compressed and pumped into one set of cylindrical aerobic digesters containing one or more iron oxidizing chemoautotrophs such as but not limited to Leptospirillum ferrooxidans or Thiobacillus ferrooxidans, and another set of cylindrical anaerobic digesters containing one or more hydrogen oxidizing acetogenic chemoautotrophs such as but not limited to Acetoanaerobium noterae, Acetobacterium woodii, Acetogenium kivui, Butyribacterium methylotrophicum, Butyribacterium rettgeri, Clostridium aceticum, Clostridium acetobutylicum, Clostridium acidi-urici, Clostridium autoethanogenum, Clostridium carboxidivorans, Clostridium formicoaceticum, Clostridium kluyveri, Clostridium ljungdahlii, Clostridium thermoaceticum, Clostridium thermoautotrophicum, Clostridium thermohydrosulfuricum, Clostridium thermosaccharolyticum, Clostridium thermocellum, Eubacterium limosum, Peptostreptococcus productus. Hydrogen gas produced by the water shift reaction is fed into the H-oxidizer digesters as the electron donor. Ferrous sulfate synthesized through the reaction of ferrous oxide (FeO), sulfur dioxide and oxygen is pumped into the Fe-oxidizer digesters as the electron donor. The other nutrients required for chemoautotrophic growth are also pumped into the digesters for each respective type of chemoautotroph. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. In the Fe-oxidizer process stream the cell-free broth which has passed through the cell mass removing filters is directed to ferric sulfate recovery systems such as employed in the steel industry where the ferric sulfate product of chemosynthetic metabolism is concentrated into a salable product. The broth left over after the sulfate has been removed is then subjected to any necessary additional waste removal treatments which depends on the source of flue gas. In the H-oxidizer process stream the cell-free broth which has passed through the cell mass removing filters is directed to vessels where the acetic acid produced is reacted with ethanol to produce ethyl acetate which is removed from solution by reactive distillation. The ethyl acetate is converted to ethanol by hydrogenation. Half of the ethanol is recycled for further reaction in the reactive distillation process. The other half is put through a molecular sieve which separates anhydrous ethanol by adsorption from dilute ethanol. The anhydrous ethanol is then collected and the dilute ethanol is returned for further reaction in the reactive distillation step. The broth left over after the acetic acid is reactively distilled out is then subjected to any necessary additional waste removal treatments which depends on the source of flue gas. The remaining water and nutrients in both process streams are then pumped back into their respective digesters. Both the hydrogen gas and ferrous sulfate electron donors are ultimately generated through the oxidation of coal or some other hydrocarbon. The oxidation drives two reactions that occur in parallel, one is the reduction of iron ore (Fe.sub2.O.sub3) to ferrous oxide (FeO) accompanied by the release of carbon monoxide which is water shifted to produce hydrogen gas and carbon dioxide, the other is the reduction of gypsum (CaSO.sub.4) to sulfur dioxide and quicklime accompanied by the release of carbon dioxide. The carbon dioxide from both process streams is reacted with the quicklime to produce calcium carbonate. In parallel with the production of calcium carbonate is the production of ferrous sulfate through the reaction of ferrous oxide with sulfur dioxide and oxygen.

It should be noted that in all of the previously described embodiments with a sulfuric acid product the sulfuric acid may alternatively be neutralized, preferably with a base that is not a carbonate (so as to release not carbon dioxide in the acid base reaction) and this is produced by a carbon dioxide emission-free process. Such preferred bases include but are not limited to natural basic minerals containing a metal oxide, serpentine containing a metal oxide, ultramafic deposits containing metal oxides, underground basic saline aquifers, and naturally occurring calcium oxide, magnesium oxide, iron oxide, or some other metal oxide. The metal sulfate which results from the acid-base reaction is recovered from the process stream and preferably refined into a salable product, while the water produced by the acid-base reaction is preferably recycled back into the chemosynthesis reactors.

### Example

An example is provided to demonstrate the carbon capture and fixation capabilities of chemoautotrophic microorganisms that play a central part in the overall carbon capture and fixation process of the present invention.

Tests were performed on the sulfur-oxidizing chemoautotroph Thiomicrospira crunogena ATCC #35932 acquired as a freeze dried culture from American Type Culture Collection (ATCC). The organisms were grown on the recommended ATCC medium—the #1422 broth. This broth consisted of the following chemicals dissolved in 1 Liter of distilled water:

### NaCl, 25.1 g

### (NH.sub.4).sub.2SO.sub.4, 1.0 g

### MgSO.sub.4.7H.sub.2O, 1.5 g

### KH.sub.2PO.sub.4, 0.42 g

### NaHCO.sub.3, 0.20 g

### CaCl.sub.2.2H.sub.2O, 0.29 g

Tris-hydrochloride buffer, 3.07 g

### Na.sub.2S.sub.2O.sub.3.5H₂O, 2.48 g

### Visniac and Santer Trace Element Solution, 0.2 ml

### 0.5% Phenol Red, 1.0 ml

The #1422 broth was adjusted to pH 7.5 and filter-sterilized prior to inoculation. The freeze dried culture of Thiomicrospira crunogena was rehydrated according to the procedure recommended by ATCC and transferred first to a test tube with 5 ml broth #1422 and placed on a shaker. This culture was used to innoculate additional test tubes. NaOH was added as needed to maintain the pH near 7.5. Eventually the cultures were transferred from the test tube to 1 liter flasks filled with 250 ml of #1422 broth and placed in a New Brunswick Scientific Co. shake flask incubator set to 25 Celsius.

The determination of growth rate for Thiomicrospira crunogena was performed using the following procedure.

1) Three (1 litre) flasks containing 95 ml ATCC 1422 medium were innoculated with 5 ml of the above cultures diluted to an optical density ˜0.025. Optical densities were determined using a Milton Roy Spectronic 1001 Spectrophotometer.
2) Two ml samples of cultures were withdrawn from each flask from t=0 to t=48 hours at every 2 hour intervals and optical density measured. Optical density was correlated with dry weight weighing twice centrifuged and washed, 1 mL liquid broth oven dried samples in pre-weighed aluminum dishes.

From the growth curve is was found that in the exponential phase the doubling time for Thiomicrospira crunogena was one hour. This is about 4 to 6 times shorter doubling time than the fastest growth rates reported for algae in the exponential phase [Sheehan et al, 1998, “A Look Back at the U.S. Department of Energy's Aquatic Species Program—Biodiesel from Algae”]. The cell mass density present in the flask experiments when the microorganisms were in the exponential growth phase reached 0.5 g dry weight/liter, and in the plateau phase the cell mass density reached 1 g dry weight/liter. This indicates that in a continuous system that maintains the culture in the exponential growth state with continuous cell removal, these microorganisms have the potential to produce 12 g dry weight/liter/day of biomass. This is about 4-12 times faster than the highest daily rates of biomass production reported for algae [Valcent, 2007; CNN, 2008]. Furthermore it is likely that in a continuous bioreactor substantially higher cell densities can be sustained in the exponential phase than what can be achieved the flask level with T. crunogena. This experiment supports the far higher rates of carbon fixation that are attainable with chemoautotrophic than photosynthetic microbes.

Specific preferred embodiments of the present invention have been described here in sufficient detail to enable those skilled in the art to practice the invention. However it is to be understood that many possible variations of the present invention, which have not been specifically described, still fall within the spirit of the present invention and the scope of its claims. Hence these descriptions given herein are added only by way of example and are not intended to limit, in any way, the scope of this invention.

What is claimed is:

1. A multistep biological and chemical process for the capture and conversion of carbon dioxide and/or other sources of inorganic carbon, into organic compounds, where one or more steps in the process utilize obligate and/or facultative chemoautotrophic microorganisms, and/or cell extracts containing enzymes from chemoautotrophic microorganisms, to fix carbon dioxide or inorganic carbon into organic compounds where carbon dioxide gas alone or in a mixture or solution as dissolved carbon dioxide, carbonate ion, or bicarbonate ion including aqueous solutions such as sea water, or in a solid phase including but not limited to a carbonate mineral, is introduced into an environment suitable for maintaining chemoautotrophic organisms and/or chemoautotroph cell extracts, which fix the inorganic carbon into organic compounds, with the chemosynthetic carbon fixing reaction being driven by chemical and/or electrochemical energy provided by electron donors and electron acceptors that have been generated chemically or electrochemically or input from inorganic sources or waste sources that are made accessible through the process to the chemoautotrophic microorganisms in the chemosynthetic reaction step or steps.

2. A method according to claim 1, whereby said electron donors include but are not limited to one or more of the following reducing agents: ammonia; ammonium; carbon monoxide; dithionite; elemental sulfur; hydrocarbons; hydrogen; metabisulfites; nitric oxide; nitrites; sulfates such as thiosulfates including but not limited to sodium thiosulfate (Na.sub.2S.sub.2O.sub.3) or calcium thiosulfate (CaS.sub.2O.sub.3); sulfides such as hydrogen sulfide; sulfites; thionate; thionite; transition metals or their sulfides, oxides, chalcogenides, halides, hydroxides, oxyhydroxides, phosphates, sulfates, or carbonates, in dissolved or solid phases; as well as conduction or valence band electrons in solid state electrode materials.

3. A method according to claim 1, whereby said electron acceptors include but are not limited to one or more of the following: carbon dioxide; oxygen; nitrites; nitrates; ferric iron or other transition metal ions; sulfates; or valence or conduction band holes in solid state electrode materials.

4. A method according to claim 1, whereby the said chemosynthetic step or steps is proceeded by one or more chemical preprocessing steps whereby said electron donors and/or said electron acceptors used to drive chemosynthesis and/or other nutrients needed to support the chemoautotrophic culture are generated or refined from more unrefined raw input chemicals and/or recycled from process output chemicals and/or the waste streams from other industrial, mining, agricultural, sewage or waste generating processes.

5. A method according to claim 1, whereby the said chemosynthetic step or steps is followed by one or more process steps for the separation of the organic and/or inorganic chemical products of chemosynthesis from the process stream and for the processing of these products into a form suitable for storage, shipping, and sale; as well as one or more process steps for the separation of cell mass from the process stream and for the recycling of cell mass needed to maintain the chemoautotrophic culture back into the said chemosynthetic steps, and/or for surplus biomass to be processed into a form suitable for storage, shipping, and sale

6. A method according to claim 1, whereby the said chemosynthetic step or steps is followed by one or more process steps where waste products and/or impurities or contaminants are removed from the process stream including the nutrient medium used to maintain the chemoautotrophic culture, and disposed of.

7. A method according to claim 1, whereby the said chemosynthetic step or steps is followed by one or more process steps where any unused nutrients and/or process water left after the removal of chemoautotrophic cell mass and/or chemical co-products of chemosynthesis and/or waste products or contaminants are recycled back into the chemosynthetic process steps to support further chemosynthesis.

8. A method according to claim 1, whereby the given chemoautotrophic microorganisms include but are not limited to one or more of the following: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Arcobacter sp.; Aureobacterium sp.; Bacillus sp.; Beggiatoa sp.; Butyribacterium sp.; Carboxydothermus sp.; Clostridium sp.; Comamonas sp.; Dehalobacter sp.; Dehalococcoide sp.; Dehalospirillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobacterium sp.; Methanobrevibacter sp.; Methanococcus sp.; Methanosarcina sp.; Micrococcus sp.; Nitrobacter sp.; Nitrosococcus sp.; Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Streptomyces sp.; Sulfobacillus sp.; Sulfolobus sp.; Thiobacillus sp.; Thiomicrospira sp.; Thioploca sp.; Thiosphaera sp.; Thiothrix sp.; sulfur-oxidizers; hydrogen-oxidizers; iron-oxidizers; acetogens; methanogens; as well as a consortiums of microorganisms that include chemoautotrophs, where the chemoautotrophs may be native to environments including but not limited to: hydrothermal vents; geothermal vents; hot springs; cold seeps; underground aquifers; salt lakes; saline formations; mines; acid mine drainage; mine tailings; oil wells; refinery wastewater; coal seams; the deep sub-surface; waste water and sewage treatment plants; geothermal power plants; sulfatara fields; soils; where the said chemoautotrophs may or may not be extremophiles including but not limited to thermophiles, hyperthermophiles, acidophiles, halophiles, and psychrophiles.

9. A method according to claim 1, whereby said electron donors and/or electron acceptors are generated or recycled using renewable, alternative, or conventional sources of power that are low in greenhouse gas emissions including but not limited to one or more of the following: photovoltaics, solar thermal, wind power, hydroelectric, nuclear, geothermal, enhanced geothermal, ocean thermal, ocean wave power, tidal power.

10. A method according to claim 1, whereby molecular hydrogen acts as electron donor and is generated through electrolysis of water including but not limited to approaches using Proton Exchange Membranes (PEM), liquid electrolytes such as KOH, high-pressure electrolysis, high temperature electrolysis of steam (HTES); and/or thermochemical splitting of water through methods including but not limited to the iron oxide cycle, cerium(IV) oxide-cerium(III) oxide cycle, zinc zinc-oxide cycle, sulfur-iodine cycle, copper-chlorine cycle, calcium-bromine-iron cycle, hybrid sulfur cycle; and/or the electrolysis of hydrogen sulfide; and/or the thermochemical splitting of hydrogen sulfide; and/or through other electrochemical or thermochemical processes known to produce hydrogen with low- or no-carbon dioxide emissions including but not limited to: carbon capture and sequestration enabled methane reforming; carbon capture and sequestration enabled coal gasification; the Kværner-process and other processes generating a carbon-black product; carbon capture and sequestration enabled gasification or pyrolysis of biomass; and the half-cell reduction of H⁺ to H₂ accompanied by the half-cell oxidization of electron sources including but not limited to ferrous iron (Fe²⁺) oxidized to ferric iron (Fe³⁺) or the oxidation of sulfur compounds whereby the oxidized iron or sulfur can be recycled to back to a reduced state through additional chemical reaction with minerals including but not limited to metal sulfides, hydrogen sulfide, or hydrocarbons.

11. A method according to claim 1, whereby said electron donors are generated from minerals of natural origin including but not limited to one or more of the following: elemental Fe.sup.0; siderite (FeCO.sub.3); magnetite (Fe.sub.3O.sub.4); pyrite or marcasite (FeS.sub.2), pyrrhotite (Fe.sub.(1-x)S (x=0 to 0.2), pentlandite (Fe,Ni).sub.9S.sub.8, violarite (Ni.sub.2FeS.sub.4), bravoite (Ni,Fe)S.sub.2, arsenopyrite (FeAsS), or other iron sulfides; realgar (AsS); orpiment (As.sub.2S.sub.3); cobaltite (CoAsS); rhodochrosite (MnCO.sub.3); chalcopyrite (CuFeS.sub.2), bornite (Cu.sub.5FeS.sub.4), covellite (CuS), tetrahedrite (Cu.sub.8Sb.sub.2S.sub.7), enargite (Cu.sub.3AsS.sub.4), tennantite (Cu.sub.12As.sub.4. S.sub.13), chalcocite (Cu.sub.2S), or other copper sulfides; sphalerite (ZnS), marmatite (ZnS), or other zinc sulfides; galena (PbS), geocronite (Pb.sub.5(Sb,As.sub.2)S.sub.8), or other lead sulfides; argentite or acanthite (Ag.sub.2S); molybdenite (MoS.sub.2); millerite (NiS), polydymite (Ni.sub.3 S.sub.4) or other nickel sulfides; antimonite (Sb.sub.2S.sub.3); Ga.sub.2S.sub.3; CuSe; cooperite (PtS); laurite (RuS.sub.2); braggite (Pt,Pd,Ni)S; FeCl.sub.2.

12. A method according to claim 1, whereby said electron donors are generated from pollutants or waste products including but are not limited to one or more of the following: process gas; tail gas; enhanced oil recovery vent gas; biogas; acid mine drainage; landfill leachate; landfill gas; geothermal gas; geothermal sludge or brine; metal contaminants; gangue; tailings; sulfides; disulfides; mercaptans including but not limited to methyl and dimethyl mercaptan, ethyl mercaptan; carbonyl sulfide; carbon disulfide; alkanesulfonates; dialkyl sulfides; thiosulfate; thiofurans; thiocyanates; isothiocyanates; thioureas; thiols; thiophenols; thioethers; thiophene; dibenzothiophene; tetrathionate; dithionite; thionate; dialkyl disulfides; sulfones; sulfoxides; sulfolanes; sulfonic acid; dimethylsulfoniopropionate; sulfonic esters; hydrogen sulfide; sulfate esters; organic sulfur; sulfur dioxide and all other sour gases.

13. A method according to claim 1, whereby the delivery of reducing equivalents from the said electron donors to the chemoautotrophs for the said chemosynthetic reaction or reactions is kinetically and/or thermodynamically enhanced through means including but not limited to: the introduction of hydrogen storage materials into the chemoautotrophic culture environment that can double as a solid support media for microbial growth—bringing absorbed or adsorbed hydrogen electron donors into close proximity with the hydrogen-oxidizing chemoautotrophs; the introduction of electron mediators such as but not limited to cytochromes, formate, methyl-viologen, NAD⁺/NADH, neutral red (NR), and quinones to help transfer reducing power from poorly soluble electron donors such as but not limited to H₂ gas or electrons in solid state electrode materials, into the chemoautotrophic culture media; the introduction of electrode materials that can double as a solid growth support media directly into the chemoautotrophic culture environment—bringing solid state electrons into close proximity with the microbes.

14. A method according to claim 1, whereby said electron donors are generated or recycled through non- or low-carbon dioxide emitting chemical reactions with hydrocarbons including but not limited to the thermochemical reduction of sulfate reaction (TSR) and the Muller-Kuhne reaction for the production of hydrogen sulfide or reduced sulfur; or methane reforming-like reactions utilizing metal oxides in place of water such as but not limited to iron oxide, calcium oxide, or magnesium oxide whereby the hydrocarbon is reacted to form solid carbonate with little or no emissions of carbon dioxide gas along with hydrogen electron donor product.

15. A method according to claim 1, whereby said chemosynthetic reaction or reactions are performed by chemoautotrophic microorganisms that have been improved, optimized or engineered for the fixation of carbon dioxide and/or other forms of inorganic carbon and the production of organic compounds through methods including but not limited to one or more of the following: accelerated mutagenesis, genetic engineering or modification, hybridization, synthetic biology or traditional selective breeding.

16. A method according to claim 1 whereby the said chemosynthetic reaction or reactions results in the formation of chemicals including but not limited to acetic acid, other organic acids and salts of organic acids, ethanol, butanol, methane, hydrogen, hydrocarbons, sulfuric acid, sulfate salts, elemental sulfur, sulfides, nitrates, ferric iron and other transition metal ions, other salts, acids or bases.

17. A method according to claim 1, whereby the organic and/or inorganic chemical products recovered from the chemoautotrophic growth medium of the said chemosynthetic reaction or reactions have applications including but not limited to: as biofuels or as feedstock for biofuel production; in the production of fertilizers; as leaching agents for the chemical extraction of metals in mining or bioremediation, as chemicals reagents in industrial or mining processes.

18. A method according to claim 1, whereby biomass and/or biochemicals produced through the said chemosynthetic reaction or reactions has applications including but not limited to: as a biomass fuel for combustion in particular as a fuel to be co-fired with fossil fuels; as a carbon source for large scale fermentations to produce various chemicals including but not limited to commercial enzymes, antibiotics, amino acids, vitamins, bioplastics, glycerol, or 1,3-propanediol; as a nutrient source for the growth of other microbes or organisms; as feed for animals including but not limited to cattle, sheep, chickens, pigs, or fish; as feed stock for alcohol or other biofuel fermentation and/or gasification and liquefaction processes including but not limited to direct liquefaction, Fisher Tropsch processes, methanol synthesis, pyrolysis, or microbial syngas conversions, for the production of liquid fuel; as feed stock for methane or biogas production; as fertilizer; as raw material for manufacturing or chemical processes; as sources of pharmaceutical, medicinal or nutritional substances; soil additives and soil stabilizers.

19. A method according to claim 1, whereby said chemoautotrophic microorganism cultures are maintained in apparatus known in the art and science of microbial culturing including but not limited to: airlift reactors; biological scrubber columns; bioreactors; bubble columns; continuous stirred tank reactors; counter-current, upflow, expanded-bed reactors; digesters and in particular digester systems such as known in the prior arts of sewage and waste water treatment or bioremediation; filters including but not limited to trickling filters, rotating biological contactor filters, rotating discs, soil filters; fluidized bed reactors; gas lift fermenters; immobilized cell reactors; membrane biofilm reactors; mine shafts; pachuca tanks; packed-bed reactors; plug-flow reactors; static mixers; tanks; trickle bed reactors; vats; vertical shaft bioreactors; wells caverns; caves; cisterns; lagoons; ponds; pools; quarries; reservoirs; towers—with the vessel base, siding, walls, lining, or top constructed out of one or more materials including but not limited to bitumen, cement, ceramics, clay, concrete, epoxy, fiberglass, glass, macadam, plastics, sand, sealant, soil, steels or other metals and their alloys, stone, tar, wood, and any combination thereof.

20. A method according to claim 1 where additional sequestration of carbon dioxide is accomplished through steps in the carbon capture and conversion process where carbon dioxide is reacted with minerals including but not limited to oxides or hydroxides to form a carbonate or bicarbonate product.
